(19) United States
(12) Patent Application Publication
(10) Pub. No.: US 2014/0314798 A1
(43) Pub. Date: Oct. 23, 2014

(54) METHOD TO MEASURE INFLAMMATION IN THE CONJUNCTIVA OF PATIENTS WITH TEAR DYSFUNCTION

(71) Applicant: BAYLOR COLLEGE OF MEDICINE, Houston, TX (US)

(72) Inventors: Stephen C. Pflugfelder, Houston, TX (US); Cintia S. De Paiva, Houston, TX (US); Rosa M. Corrales Herran, Houston, TX (US); De-Quan Li, Houston, TX (US)

(21) Appl. No.: 14/353,283

(22) PCT Filed: Oct. 19, 2012
(86) PCT No.: PCT/US12/61095

§ 371 Date: Apr. 21, 2014

Related U.S. Application Data
(60) Provisional application No. 61/549,781, filed on Oct. 21, 2011.

(51) Int. Cl. C12Q 1/68

(52) U.S. Cl. CPC C12Q 1/686
USPC 424/184.1; 435/6.12; 514/21.1; 514/152; 514/179

(57) ABSTRACT

The present invention concerns methods and compositions for treatment and determination of the presence or likelihood of an individual to have an ocular surface inflammation. In specific embodiments, a sample from the individual is assayed for the expression level of three or more genes, including IL-6, MMP3, MMP9, IFNy, SPRR-IA, HLA-DRA, MUC5AC, K7, and IL17A. Treatment is provided to an individual with ocular surface inflammation based upon the gene analysis.

This application claims priority to U.S. Provisional Patent Application Ser. No. 61/549,781, filed Oct. 21, 2011, which application is incorporated by reference herein in its entirety.

### STATEMENT REGARDING FEDERALLY SPONSORED RESEARCH OR DEVELOPMENT

This invention was made with government support under EY11915 awarded by the National Institutes of Health, National Eye Institute. The government has certain rights in the invention.

### TECHNICAL FIELD

The field of the invention generally concerns at least the fields of cell biology, molecular biology, and medicine, including opthalmological medicine.

### BACKGROUND OF THE INVENTION

The cornea is a unique optically clear tissue, devoid of blood vessels, that relies on tears to maintain a moist, smooth, and lubricated surface in the face of near-constant exposure to ambient environmental conditions during waking hours. Additionally, the tears provide myriad factors that protect the cornea from microbial infection and the sight-threatening effects of excessive inflammation or prolonged wound healing. To maintain corneal clarity and quality vision, humans have a complex and highly regulated system to produce and distribute tears.

Tear dysfunction is one of the most prevalent medical conditions, affecting tens of millions of patients worldwide. Tear dysfunction is a more encompassing term than dry eye for tear-associated disorders of the ocular surface and cornea because it encompasses changes in tear composition rather than tear volume. (Behrens et al., 2006) Tear dysfunction has long been recognized to cause corneal epithelial disease that can decrease visual performance and cause ocular irritation. Mechanisms responsible for these pathologic changes were poorly understood until evidence from recent clinical studies and animal models indicated that altered tear composition causes dysfunction, accelerated death, and detachment of the superficial epithelium, leading to an irregular corneal surface, an unstable tear layer, and hyperesthesia of the corneal nerve endings. These changes in the superficial cornea can significantly impact quality of life and productivity in patients suffering from tear dysfunction.

### BRIEF SUMMARY OF THE INVENTION

Embodiments of the invention concern treatment of ocular surface inflammation. The treatment is established following determination of gene expression analysis of one or more genes from conjunctival cells, such as in vivo from the eye. Treatment for the ocular surface inflammation may have been unsuitable in the absence of determination of the particular gene expression analysis. The cells may be obtained by any method, but in particular cases the cells are obtained upon direct contact with the eye surface, such as with an instrument that encompasses a substrate for the contact. The substrate may be pressed against the eye surface, thereby extracting conjunctival cells from the eye surface for analysis.

Embodiments of the present invention concern at least the use of one or more testing method(s) to identify genes in relation to the presence, onset, or risk of developing one or more ocular diseases, including medical conditions associated with the ocular surface, such as at least ocular surface inflammation. In some embodiments the methods of the invention employ analysis of conjunctival cells (and optionally sampling of the conjunctival cells) for one or more biomarkers associated with ocular surface medical conditions. In some embodiments, one utilizes cells obtained by touching the surface of the eye in vivo, such as with a membrane, a brush, and/or washing. Conjunctival cells obtained from an in vivo eye are processed by routine methods to obtain RNA, after which the copy number of expression of one or more particular genes is determined. The quantitative results of the methods are utilized to ascertain the levels of expression of one or more particular genes. In specific embodiments, the copy number level is within a range that is indicative whether or not an individual has or is at risk of having an ocular disease. In some cases, the symptoms of an individual are monitored in addition to the initial determination and/or monitoring of gene expression (such as by copy number) of one or more particular genes.

Embodiments of the present invention are directed to methods and compositions that concern diagnosis/prognosis of individuals with ocular surface (including at least corneal) disease, including inflammation, tear dysfunction-related corneal, conjunctival and/or lid margin disease, in which modulation of the expression level of one or more genes is associated with onset of the disease, risk for developing the disease, identification of the stage and/or severity of the disease, indications for therapy, and/or progress of the disease, including following therapy, for example. In some embodiments, the gene level that is assayed is for matrix metalloproteinase (MMP) 3, MMP9, IFNγ, IL17A, IL-6, HLD-DRA, MUC5AC, K7, IL-13, or a combination thereof, although in some embodiments one or more other gene expression levels are also determined. The individual subjected to methods and compositions of the invention is a male or female mammal, in certain aspects, including a human, dog, cat, horse, and so forth.

In specific embodiments, a diagnostic test is provided to measure levels of inflammation in individuals who present to medical providers with one or more symptoms or signs of ocular surface disease, such as one or more irritation symptoms and or clinical signs of tear dysfunction, for example. In particular aspects of the invention, inflammation is a major contributor to development of surface epithelial disease in the cornea and conjunctiva of the eye. In some embodiments, there is a role of various inflammatory mediators in the development of corneal and conjunctival epithelial disease and tear dysfunction related eye discomfort. Detection of increased levels of inflammation with methods of the invention is useful to medical providers for several reasons: 1) it identifies individuals that should be treated with anti-inflammatory treatments, 2) based on the cytokine profile, it indicates which agents are most effective; and/or 3) it identifies individuals at risk for developing complications from ocular surgeries, such as LASIK, where worsening dry eye would reduce visual outcome.

In certain embodiments of the invention, there is a method of treating an individual for an ocular surface medical condition comprising the step of delivering to the individual a suitable treatment for the medical condition, wherein the method comprises the step of determining the expression level of one or more particular genes. In certain embodiments of the invention, there is a method of treating an individual for an ocular surface medical condition comprising the step of delivering to the individual a suitable treatment for the medical condition, wherein the suitable treatment is realized upon determination of the expression level (including copy number) of one or more particular genes. In specific embodiments, the suitable treatment is unknown until the expression level of one or more particular genes is determined. The determination of the expression level may occur at the point of examination of an individual for a medical condition wherein a suitable treatment follows the determination and is provided directly or indirectly by the medical provider. For example, in some cases an individual with one or more ocular surface inflammatory medical condition symptoms is considered an indication for treatment for an ocular surface medical condition wherein the treatment may be one or more anti-inflammatory compositions, one or more immunomodulatory compositions, steroids, or antibiotics. The medical provider may desire to avoid incorrectly prescribing steroids (for deleterious side effects, for example) or avoid incorrectly prescribing antibiotics (because overuse leads to resistance) and will employ one or more methods of the invention prior to treatment.

In specific embodiments, the methods employ a quantitative determination of gene transcript copy number that dictates at least in part the presence or risk of developing an ocular surface inflammation. Any suitable statistical quantitative analysis means may be employed. In specific embodiments, if the value of the copy number for expression of a particular gene from a sample of an individual is above the 75th percentile value of a control group, then the individual has or is at risk for developing ocular surface inflammation. Treatment of the disease is thereby indicated, particularly when the individual has one or more signs or symptoms of the disease that, if untreated, renders the individual at risk for increasing the severity of one or more signs or symptoms of the disease. In cases wherein the risk of developing ocular surface inflammation is determined based on the copy number analysis, the individual may be subjected to preventative measures or may be at least informed upon onset of one or more symptoms for ocular surface inflammation.

In certain embodiments, there are threshold levels of expression of one or more genes above or below which is considered abnormal. In certain cases, there is a direct correlation between levels of particular transcripts, such as mediator transcripts, and clinical severity. In specific embodiments, individuals with the most severe disease (level 3 or, in some cases, level 2) have the highest levels of certain mediators. In certain aspects, the invention is employed to evaluate individuals that are candidates for enrollment into therapeutic clinical trials.

Embodiments of the invention concern ocular surface inflammation, but in particular aspects the ocular surface inflammation concerns tear dysfunction. In some embodiments of the invention, the invention addresses the effects of tears on maintaining ocular surface health, the impact of tear dysfunction and its associated inflammation on the ocular surface, and consequences of tear dysfunction-related ocular surface disease on patient well-being. In specific embodiments of the invention, there are methods and compositions for treatment of the causes of tear dysfunction-related corneal disease.

In certain embodiments of the invention, methods and compositions concern identification of ocular surface inflammation as the cause or consequence of tear dysfunction. Such tear deficiency or dysfunction may be the result of aqueous tear deficiency (ATD), in some cases. In specific embodiments of the invention, the methods and compositions regard identification of keratoconjunctivitis sicca (KCS), including Sjögren syndrome (SS)-associated KCS and non-SS associated KCS. Individuals with aqueous tear deficiency suffer from SS if they have associated xerostomia and/or connective tissue disease, and in some aspects the invention is applicable to primary or secondary SS. The skilled artisan recognizes that non-SS KCS is mostly found in postmenopausal women, in pregnant women, in women on oral contraceptives, or in women on hormone replacement therapy. In specific embodiments, the individuals have a decrease in androgens, for example. In some aspects of the invention, an individual has a history of arthritis, gout or usage of corticosteroids, antidepressants can lead to eye dryness. In specific cases, an individual has no obvious predisposition to having dry eye or tear dysfunction.

In certain aspects of the invention, tear film dysfunction and dry eye syndrome are synonymous.

In specific embodiments of the invention, an individual has one or more of the following defects: 1) improper tear composition; 2) decreased aqueous tear production; 3) excessive tear evaporation; and/or 4) abnormality in lipid components or mucin in the tear production.

In some methods of the invention, one can obtain a sample from the individual to be assayed in methods and/or by compositions of the invention, and the inventive methods may or may not be performed by the individual that obtained the sample. The sample may be obtained by another party and the obtaining in methods of the invention comprises retrieving the sample from a repository or collection holding device, for example.

In some embodiments, genes other than (MMP) 3, MMP9, IFNγ, IL17A, IL-6, HLD-DRA, MUC5AC, K7, IL-13, or a combination thereof are utilized alternatively or in addition to these genes. In specific embodiments, the expression level of IL-6, IL-13, SPRR1A, SPRR2A, SPRR2B, SPRR2F and/or SPRR2G assayed in methods of the invention. In specific embodiments, once can discriminate between normal controls as well as between aqueous tear deficiency and meibomian gland disease with one or more of IL-6, IL-13, SPRR1A, SPRR2A, SPRR2B, SPRR2F and SPRR2G.

In one embodiment of the invention, there is a method of identifying an individual that has, or is at risk for having, tear dysfunction, comprising the step of assaying the expression level of one, two, three, four, five, or more genes selected from the group consisting of (MMP) 3, MMP9, IFNγ, IL17A, IL-6, HLD-DRA, MUC5AC, K7, IL-13, or a combination thereof from a sample from the individual. In a specific embodiment, the expression level is determined by mRNA level, protein level, or both.

In specific embodiments of the invention, when the expression level of the three or more genes selected from the group consisting of (MMP) 3, MMP9, IFNγ, IL17A, IL-6, HLD-DRA, MUC5AC, K7, IL-13, or a combination thereof is elevated compared to a reference, the individual has, or is at risk for having, tear dysfunction and tear dysfunction-associated ocular surface inflammation.

In some embodiments, the individual is subjected to another method of identifying if the individual has or is at risk for having tear dysfunction and tear dysfunction associated ocular surface inflammation.

In some embodiments, when the individual is identified as having tear dysfunction, the individual is subjected to one or more treatments for tear dysfunction.

In specific cases, the methods further comprise the step of obtaining a sample from the individual.

In an embodiment of the invention, there is a kit for assaying an individual for the presence or risk for tear dysfunction and tear dysfunction associated ocular surface inflammation, comprising one or more reagents suitable for determining gene expression level of at least three genes selected from the group consisting of (MMP) 3, MMP9, IFNγ, IL17A, IL-6, HLD-DRA, MUC5AC, K7, IL-13, or a combination thereof.

In embodiments of the invention, there is a method of treating an individual for ocular surface inflammation, comprising the steps of providing a therapeutically effective amount of a suitable treatment for the ocular surface inflammation to the individual, said treatment resultant upon determination of expression level of one or more genes selected from the group consisting of MMP3, MMP9, IFNγ, IL17A, IL-6, HLD-DRA, MUC5AC, K7, IL-13, and a combination thereof. In specific embodiments, when the expression level of IL-17A is elevated compared to a reference, the individual is treated with a T cell immunosuppressant. In some cases, when the expression level of IFN-γ is elevated compared to a reference, the individual is treated with a T cell immunosuppressant. In certain aspects, when the expression level of MMP3 is elevated compared to a reference, the individual is treated with an antiprotease. In certain aspects, when the expression level of MMP9 is elevated compared to a reference, the individual is treated with an antiprotease. In some cases, when the expression level of IL-6 is elevated compared to a reference, the individual is treated with an anti-inflammatory. In particular embodiments, when the expression level of HLA-DRA is elevated compared to a reference, the individual is treated with an anti-inflammatory.

In particular aspects, the individual has one or more symptoms selected from the group consisting of dry eye, red eye, improper tear composition, decreased aqueous tear production, excessive tear evaporation, abnormality in lipid components or mucin in the tear production, tear instability, and a combination thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1. Agarose gel showing the size of products from conventional PCR.

FIG. 2. Melt curve for MMP-3.

FIG. 3. Melt curve for MMP-9.

FIG. 4. Melt curve for IL-17A.

FIG. 5. Melt curve for IFN-γ.

FIG. 6. Amplification plot and the standard curve for MMP-3. The amplification plot represents ΔRn (The magnitude of normalized fluorescence signal generated by the SYBR Green at each cycle during the PCR amplification) vs Cycle. The standard curve plot represents number of threshold cycle (C_{T}) vs the quantity (log) for samples designated as standards (from 10⁶ to 10).

FIG. 7. Amplification plot and the standard curve for MMP-9. The amplification plot represents ΔRn (The magnitude of normalized fluorescence signal generated by the SYBR Green at each cycle during the PCR amplification) vs Cycle. The standard curve plot represents number of threshold cycle (C_{T}) vs the quantity (log) for samples designated as standards (from 10⁶ to 10³).

FIG. 8. Amplification plot and the standard curve for IL-17A. The amplification plot represents ΔRn (The magnitude of normalized fluorescence signal generated by the SYBR Green at each cycle during the PCR amplification) vs Cycle. The standard curve plot represents number of threshold cycle (C_{T}) vs the quantity (log) for samples designated as standards (from 10⁶ to 10).

FIG. 9. Amplification plot (9A) and the standard curve (9B) for IFN-γ. The amplification plot represents ΔRn (The magnitude of normalized fluorescence signal generated by the SYBR Green at each cycle during the PCR amplification) vs Cycle. The standard curve plot represents number of threshold cycle (C_{T}) vs the quantity (log) for samples designated as standards (from 10⁶ to 10).

FIG. 10. Melt curve for MMP-3 in standards, controls and patients (Tm=78.8). FIG. 10A is the melt curve for MMP3 in standards and 10B is the melt curve for MM3 in controls and patients together. Those figures show that the results are the same in both groups (the samples with concentration know (ST) and the samples used for the diagnostic (C+P)).

FIG. 11. Melt curve for MMP-9 in standards, controls and patients (Tm=78). FIG. 11A is the melt curve for MMP9 in standards and 11B is the melt curve for MM9 in controls and patients together. Those figures show that the results are the same in both groups (the samples with concentration know (ST) and the samples used for the diagnostic (C+P))

FIG. 12. Melt curve for IL-17A in standards, controls and patients (Tm=76). FIG. 12A is the melt curve for IL-17A in standards and 12B is the melt curve for IL-17A in controls and patients together. Those figures show that the results are the same in both groups (the samples with concentration know (ST) and the samples used for the diagnostic (C+P)).

FIG. 13. Melt curve for IFN-γ in standards, controls and patients (Tm=80). FIG. 13A is the melt curve for IFN-γ in standards and 13B is the melt curve for IFN-γ in controls and patients together. Those figures show that the results are the same in both groups (the samples with concentration know (ST) and the samples used for the diagnostic (C+P)).

FIG. 14. Agarose gel showing the correct size for the three different PCR products: standards (lane 2), controls (lane 3) and patients (lane 4). Line 1: 100 bp DNA ladder. 10 fragments from 100 bp to 1,000 bp, in 100 bp increments.

FIG. 15. Comparison of samples from patients with tear dysfunction (Sjögren's syndrome and non-Sjögren's syndrome) compared to normal controls. Mean±SEM

FIG. 16. Receiver operating curves (ROC) curves and their areas.

FIGS. 17A and 17B provide exemplary diagnostic results for IL-17A mRNA using nine controls and 20 exemplary patients. FIG. 17C shows an exemplary ROC curve for IL-17A mRNA studies.

FIG. 18A shows diagnostic results for IL-6 mRNA. FIG. 18B shows an exemplary ROC curve for IL-6 mRNA. FIG. 18C shows exemplary diagnostic results as a histogram of the frequency distribution of IL-6 mRNA. FIG. 18D demonstrates an exemplary amplification plot for IL-6. FIG. 18E shows an exemplary standard curve for IL-6. FIG. 18F shows an exemplary melt curve in standards for IL-6. FIG. 18G demonstrates an exemplary melt curve in controls and patients for IL-6.

FIG. 19A shows diagnostic results for IFN-γ mRNA. FIG. 19B shows an exemplary ROC curve for IFN-γ mRNA.

FIG. 20A demonstrates diagnostic results for MMP-3 mRNA. FIG. 20B shows an exemplary ROC curve for MMP-3 mRNA. FIG. 20C shows exemplary diagnostic results as a histogram of the frequency distribution of MMP-3 mRNA. FIG. 20D demonstrates the repeatability for testing MMP-3 levels in particular patients. FIG. 20E illustrates the repeatability for testing MMP-3 levels in particular patients as demonstrated by a line graph. FIG. 20F shows a scattergraph demonstrating the repeatability of utilizing MMP-3 levels in certain patients.

FIG. 21A shows diagnostic results for MMP-9 mRNA. FIG. 21B shows an exemplary ROC curve for MMP-9 mRNA. FIG. 21C demonstrates exemplary diagnostic results as a histogram of the frequency distribution of MMP-9 mRNA.

FIG. 22A provides diagnostic results for HLA-DRA mRNA. FIG. 22B shows an exemplary ROC curve for HLA-DRA mRNA. FIG. 22C shows an amplification plot for HLA-DRA. FIG. 22D provides an exemplary standard curve for HLA-DRA. FIG. 22E shows an exemplary melt curve in standards for HLA-DRA. FIG. 22F provides an exemplary melt curve in controls and patients for HLA-DRA.

FIG. 23A shows diagnostic results for SPRR-1A mRNA. FIG. 23B shows an exemplary ROC curve for SPRR-1A mRNA. FIG. 23C shows an exemplary amplification plot for SPRR-1A. FIG. 23D shows an exemplary standard curve for SPRR-1A.

FIGS. 24A and 24B demonstrate exemplary copy numbers of MUC5AC mRNA (FIG. 24A) in human conjunctival samples and (cytokeratin 7) K7 mRNA in human conjunctival samples (FIG. 24B).

FIG. 25 demonstrates the ratio of expression of IL-13/IFN-γ and the ratio of MUC5AC/K7.

Exemplary quality control measures are provided in FIGS. 26 and 27. FIG. 26 illustrates the exemplary control HPRT-1 mRNA between controls and patients. FIG. 27 illustrates the exemplary control 18S mRNA between controls and patients.

FIG. 28 shows an exemplary real time PCR amplification plot and the standard curve.

FIG. 29 illustrates an exemplary melt curve to verify the identity of each gene amplification product.

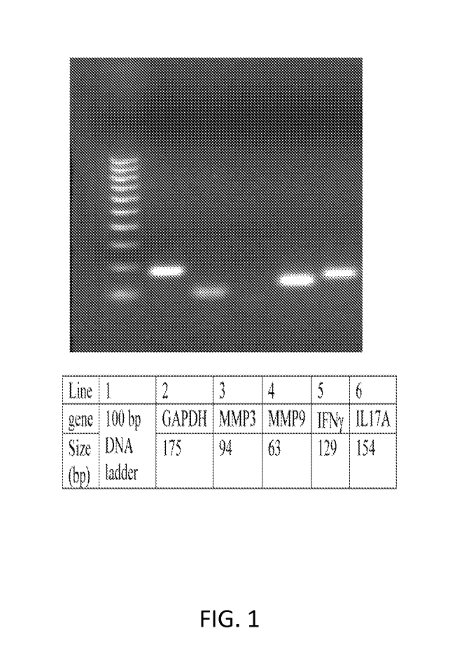

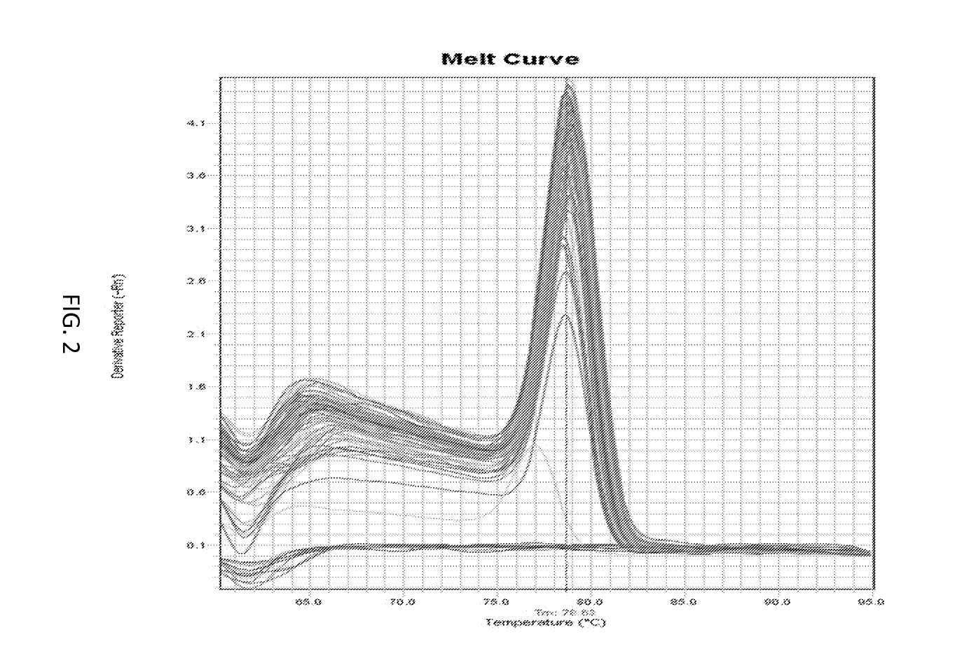

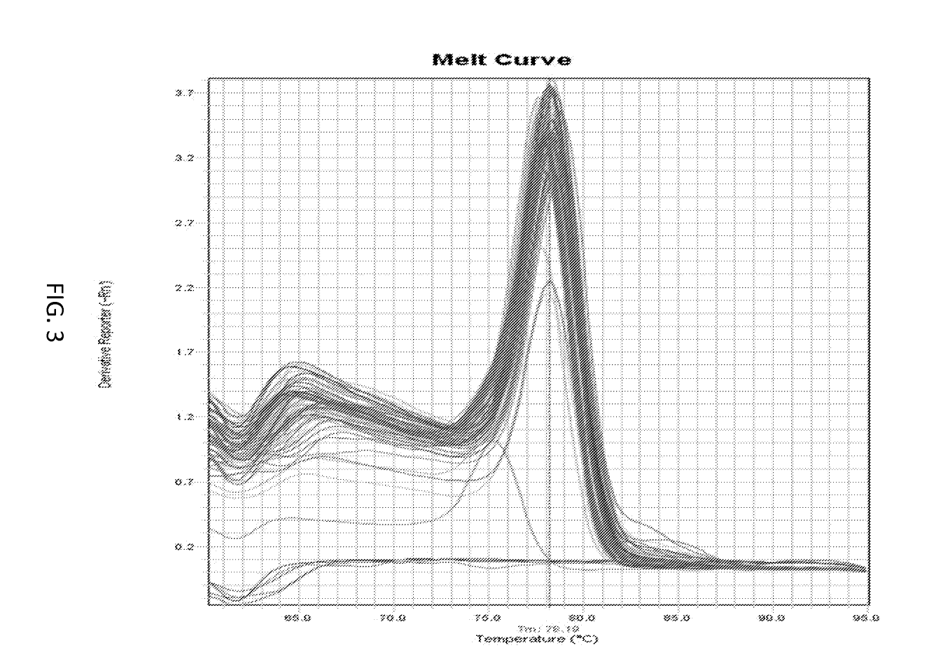

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. Furthermore, as used herein, the terms “including”, “containing”, and “having” are open-ended in interpretation and interchangeable with the term “comprising”.

The term “copy number” as used herein refers to the number of mRNA molecules (either literally or as a quantitative representation) corresponding to expression of a particular gene.

The term “ocular surface inflammation” as used herein refers to clinical of biochemical evidence of inflammation in the cornea, conjunctival, eyelid margins or tears of an individual.

General embodiments of the invention concern treatment of one or more diseases that result directly or indirectly from alterations at the molecular level of DNA, RNA, and/or proteins related to ocular surface physiology. A sample is obtained from an individual that has the disease, is suspected of having the disease (such as based on at least one sign or symptom), or is at risk for having the disease (based on at least one risk factor, such as age over 40, being a female, use of contact lenses, lower dietary consumption of n-6 to n-3 essential fatty acids, diabetes mellitus, systemic lupus erythematosis, scleroderma, rheumatoid arthritis, Sjögren syndrome, graft versus host disease, Stevens-Johnson syndrome, cigarette smoking, prolonged video display viewing and/or low-humidity environments. The sample is analyzed based on embodiments of the invention related to detection and/or quantification, and the individual is treated based on the analysis.

Embodiments of the invention are directed to ocular surface inflammation encompassing, for example, corneal epithelial disease resulting from tear dysfunction that causes eye irritation and decreases visual function. Tear dysfunction is a prevalent eye disease and the most frequent cause for superficial corneal epithelial disease that results in corneal barrier disruption, an irregular optical surface, light scattering, optical aberrations, and exposure and sensitization of pain-sensing nerve endings (nociceptors). Tear dysfunction-related corneal disease causes irritation and visual symptoms such as photophobia and blurred and fluctuating vision that may decrease quality of life. Dysfunction of one or more components of the lacrimal functional unit (conjunctival, lacrimal glands, meibomian glands) results in changes in tear composition, including elevated osmolarity and increased concentrations of matrix metalloproteinases, inflammatory cytokines, and chemokines. These tear compositional changes promote disruption of tight junctions, alter differentiation, and accelerate death of corneal epithelial cells.

Biological samples used with embodiments of the present invention may be tissue or fluid samples from a subject. Various tissues and fluids are usable, such as ocular tissue, aqueous humor, tears, blood, skin, cheek epithelial tissue, and various other tissue and fluid combinations not specifically named herein. In preferred embodiments of the present invention, biological samples are ocular tissues or ocular fluids such as tears or aqueous humor. In certain embodiments, cellular or solid material contained in fluid biological samples is first removed using centrifugation, flow cytometry or other techniques known to those of skill in the art. The separated cellular material is then itself used as a biological sample.

Biological samples may be used directly in embodiments of the invention, or may be processed prior to or during use using techniques disclosed herein or known to those of skill in the art. Processing may include, but is not limited to, isolation of nucleic acids such as DNA, RNA and derivatives thereof, protein isolation, impurity removal, etc. using methods known to those of skill in the art.

A number of different methods can be used in embodiments of the present invention to measure the expression level of the matrix metalloproteinase (MMP) 3, MMP9, IFNγ, and/or IL17A gene(s). Nucleic acid-based techniques such as nucleotide sequencing, single strand conformational polymorphism (SSCP) analysis, restriction fragment length polymorphism (RFLP) analysis, PCR, RT-qPCR, allele-specific PCR, chip-based analysis, flow cytometry, enzyme-linked immunosorbent assays (ELISA), and other quantitative and qualitative measuring techniques alone or in various combinations can be used to measure expression of the respective gene in embodiments of the present invention.

In general, embodiments of the invention incorporating techniques such as PCR, nucleotide sequencing, and/or labeled and unlabeled probe detection that rely on nucleic acid hybridization can use oligonucleotides that hybridize to all or a portion of a nucleic acid molecule with a nucleotide sequence substantially homologous to the cDNA sequence of the genes. Hybridization to nucleotide sequences related to the respective cDNA sequence, such as mRNA or other nucleotides, is also contemplated by embodiments of the present invention. Such oligonucleotides may comprise DNA, RNA, cDNA, protein nucleic acid (PNA), genomic DNA, or synthetic oligonucleotides and may be labeled or unlabeled. Oligonucleotide labels can be any of a number of labels known to those of skill in the art, such as digoxigenin, radioisotopes, and fluorescent molecules.

Nucleic acid amplification techniques used in certain embodiments of the present invention generally contact a biological sample with at least one primer consisting essentially of a nucleic acid sequence encoding part or all of the sequence of matrix metalloproteinase (MMP) 3, MMP9, IFNγ, IL17A IL-6, HLD-DRA, MUC5AC, K7, IL-13, SPRR-1A gene products (such as the cDNA). Multiple copies of a desired nucleic acid sequence or sequences present in the biological sample are then produced by action of polymerase enzyme together with free nucleotides and cofactors under controlled temperature conditions.

Nucleic acid chip-based methods used in certain embodiments of the present invention generally comprise contacting a biological sample with a solid support-bound nucleic acid molecule that hybridizes under stringent conditions to a nucleotide having a sequence substantially homologous to the sequence of the respective gene product and detecting hybridization to measure the expression level of the respective gene. In some embodiments, the solid support is a microarray, for example.

The respective nucleotide GenBank® Accession numbers include the following: NM_{—}002422 for MMP3; NM_{—}004994 for MMP9; NM_{—}000619 for IFNγ; and NM_{—}002190 for IL17A, NM_{—}000600 for IL-6, NM_{—}019111 for HLA-DRA, for NM_{—}005987 SPRR-1A, XM_{—}003403450 for MUC5AC, NM_{—}005556 for K7 all of which are incorporated herein by reference in their entirety.

The respective polypeptide GenBank® Accession numbers include the following: NP_{—}002413.1 for MMP3; NP_{—}004985.2 for MMP9; NP_{—}000610.2 for IFNγ; NP_{—}002181.1 for IL17A, NP_{—}000591.1 for IL-6, NP_{—}061984.2 for HLA-DRA, NP_{—}001186757.1 for SPRR-1A, CAC44892 for MUC5AC, NP_{—}005547 for K7, all of which are incorporated herein by reference in their entirety.

Protein-based methods may be used to measure gene expression levels in other embodiments of the present invention. In one embodiment, gene expression is measured by measuring protein levels of a polypeptide substantially homologous to the sequence of the respective protein. Measuring such protein levels can be accomplished using methods known to those of skill in the art such as ELISA, capillary electrophoresis, Western blot, immunochromatography, mass spectroscopy, immunohistochemistry, flow cytometry, Luminex's XMAP® immunobead technology, and/or protein chip assays, for example.

Protein-based methods used with certain embodiments of the present invention to measure gene expression levels may employ antibodies to the respective expressed protein. Such antibodies may include, but are not limited to, polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Antibodies used may be reactive to protein or nucleic acids, and may be produced using any of a number of antibody production methods known to those of skill in the art.

Other embodiments of the present invention comprise kits for diagnosing or monitoring tear dysfunction. In general, kits comprise a probe set (most often comprising a cDNA, oligonucleotide, or antibody) and reagents for performing an expression measurement assay using expression profiling technology known to those of skill in the art, packaged in a suitable container. The kit may further comprise one or more additional reagents such as substrates, intermediates, labels, primers, tubes and/or other accessories. Such kits may also comprise reagents for collecting blood samples, buffers, extraction reagents, hybridization chambers, etc. Certain embodiments include a software package to perform such functions as data collection and/or analysis, formatting, database accession, security, etc. The kit optionally further comprises an instruction set or user manual detailing preferred methods of using one or more of the kit components for measuring expression levels of gene to diagnose or monitor tear dysfunction.

Such kits may use array-based methods where the probe set is immobilized on an array such as a chip array, a plate array, a bead array, a pin array, a membrane array, a solid surface array, a liquid array, an oligonucleotide array, polynucleotide array or a cDNA array, a microtiter plate, or a membrane and a chip.

I. Tear Dysfunction and the Ocular Surface

Tear dysfunction occurs when the lacrimal functional unit is no longer able to maintain a stable precorneal tear layer. It may develop from dysfunction or disease of one or more components of the lacrimal functional unit. Tear dysfunction is one of the most prevalent eye conditions.

A number of risk factors for tear dysfunction/dry eye have been identified. Age is perhaps the biggest risk factor, with the prevalence increasing in both men and woman with every decade of life over the age of 40, with a greater prevalence in women than men at every age. (Schaumberg et al., 2003; Schaumberg et al., 2009) Other risk factors include use of contact lenses, (Shimmura et al., 1999; Uchino et al., 2008) lower dietary consumption of n-6 to n-3 essential fatty acids, (Miljanovic et al., 2005) diabetes mellitus, (Moss et al., 2000; Moss et al., 2004) cigarette smoking, (Mos et al., 2000; Altinors et al., 2006) prolonged video display viewing, (Uchino et al., 2008) and low-humidity environments (Uchiyama et al., 2007), for example. Individuals with tear dysfunction typically report irritation symptoms including, for example, foreign body sensation, burning, and dryness, as well as vision-related symptoms such as photophobia and blurred and fluctuating vision, for example.

The majority of the symptoms of tear dysfunction result from corneal epithelial disease. Tear dysfunction has been recognized for over a century as the major cause of superficial corneal epithelial disease. (Pfluger, Klin Monatsbl Augenheilkd 1882; 20:69-81). It is now recognized that this epitheliopathy reduces corneal barrier function, causing an irregular optical surface, light scattering, optical aberrations, and exposure and sensitization of corneal nociceptors.

II. Combination Methods for Identification of Tear Dysfunction-Related Ocular Surface Disease

In some embodiments of the invention, methods and/or compositions of the invention are employed by a practitioner as a sole means to identify an individual that has tear dysfunction-related ocular surface (cornea, conjunctiva, lacrimal gland) disease or is at risk for developing tear dysfunction-related corneal disease. However, in certain embodiments of the invention, methods and/or compositions of the invention are utilized with one or more other methods and/or compositions to identify an individual that has tear dysfunction-related ocular surface disease or is at risk for developing tear dysfunction-related ocular surface disease. The additional method may be employed either before, after, or during the timing of the methods/compositions of the invention and/or may employ the same sampling (where appropriate) as those used for the methods/compositions of the invention.

In addition to or as part of methods of the invention, one can employ histology, immunohistochemistry, scanning electron microscopy, and/or gene expression analysis (for example by using TaqMan gene assay technology).

In some embodiments, one can employ the Ocular Surface Disease Index (OSDI) in addition to methods of the invention. The OSDI is a 12-item scale for the assessment of symptoms related to dry eye disease and their effect on vision.

In certain aspects, the methods of the invention are used in conjunction with the use of one or more dyes for staining of the cornea and/or conjunctiva for evaluation of ocular surface changes in patients with dry eye syndrome. These include fluorescein, rose bengal, and/or lissamine green B, for example. (see Yoon et al., 2011, for example).

III. Treatment of Ocular Surface Inflammation Disease

In embodiments of the invention, an individual is treated for an ocular surface inflammation such as tear dysfunction-related ocular surface disease. Such treatment occurs following exposure to methods and compositions of the invention for identifying individuals having or at risk of having the disease.

Increased knowledge regarding the cellular and molecular mechanisms of tear dysfunction-mediated ocular surface epithelial disease has prompted use of therapies that target disease-related factors including topical use of targeted immunomodulators. Over the past decade there has been a trend toward increased use of anti-inflammatory therapies to improve comfort, corneal smoothness, and barrier function and conjunctival mucin production.

In specific embodiments of the invention, a treatment for an ocular surface inflammation comprises a T cell immunosuppressant, such as Cyclosporin (Restasis®), Antiprotease, such as corticosteroid or tetracycline, or anti-inflammatory, such as cyclosporin, corticosteroid, omega-3,6 supplement, for example.

Cyclosporin A (CsA), the only FDA-approved therapy for tear dysfunction, inhibits T-cell activation and production of the Th cytokines IFN-γ and IL-17 (De Paiva et al., 2011; Zhang et al., 2008) Topical CsA significantly reduced severity of corneal fluorescein staining after 4 and 6 months of use. (Sall et al., 2000) Corticosteroids, tetracyclines, and n-3/n-6 essential fatty acids have also been found to decrease production of a variety of inflammatory mediators and improve corneal epithelial disease. (Marsh and Pflugfelder, 1999; Pflugfelder et al., 2004; De Paiva et al., 2006; Li et al., 2004; Akpek et al., 1997; Rashid et al., 2008) The efficacy of corticosteroids and tetracyclines on corneal barrier function may be attributable to their ability to inhibit MMP activity. (De Paiva et al., 2006; Li et al., 2004; Smith et al., 2008) Compounds that inhibit leukocyte migration into the ocular surface tissues in dry eye, such as integrin α4β1 (VLA-4) or chemokine receptor 2 (CCR2) antagonists, were found to improve corneal barrier function in animal models of dry eye. (Ecoiffier et al., 2008; Goyal et al., 2009)

For severe corneal epitheliopathy from tear dysfunction, the prosthetic replacement of the ocular surface ecosystem (PROSE), a specially designed scleral-bearing contact lens with a fluid-filled reservoir over the cornea, has proven to be an excellent option for improving irritation symptoms and visual acuity. (Romero-Rangel et al., 2000; Rosenthal and Croteau, 2005) The fluid-filled reservoir shields the cornea from blink trauma, noxious environmental stimuli, and inflammatory mediators in the tears. The body-temperature saline reservoir also prevents corneal cooling and nerve firing that occurs in the inter-blink intervals. Patients may experience almost immediate relief in photophobia and irritation symptoms after placing the device on the cornea. Compared to artificial tears, autologous serum (20%) was found to significantly improve corneal epithelial disease in patients with severe dry eye. (Kojima et al., 2005) Injection of botulinum toxin A in the lid has been found to decrease blink force and to improve superior limbic keratoconjunctivitis and filamentary keratitis. (Chun and Kim, 2009; Mackie, 2004)

In some embodiments, treatment can include warming of the eye, such as utilizing a warming device, for example for meibomian gland dysfunction or correcting the tear breakup time.

Over-the counter tear supplements can be employed to provide hydration and lubrication to the ocular surface. The chemicals of the supplements combine with the patient's tear layer to render protection. In some cases propylene glycol demulcents and polymers of polythelene glycol 400, with polymer hydroxypropyl gear, may be employed; it acts as a gelling agent to protect the ocular surface environment. The whole treatment is very effective in reducing the signs and symptoms of dry eye.

In specific cases one can employ topical cyclosporine ophthalmic emulsion, which can be used for tear production that is suppressed due to ocular inflammation; the therapy heals the lymphocytic inflammatory response associated with DES that occurs on the ocular surface or the lacrimal glands.

In some cases, one can employ tear substitutes, like colustrum, or cyclosporine A (CsA) as a topical treatment.

### EXAMPLES

The following examples are included to demonstrate some embodiments of the invention. It should be appreciated by those of skill in the art that the techniques disclosed in the examples which follow represent techniques discovered by the inventor to function well in the practice of the invention, and thus can be considered to constitute some modes for its practice. However, those of skill in the art should, in light of the present disclosure, appreciate that many changes can be made in the specific embodiments which are disclosed and still obtain a like or similar result without departing from the spirit and scope of the invention.

### Example 1

### Conjunctival Cell Collection by Impression Cytology

Conjunctival cells for molecular assays were obtained by placing two 4×10 mm rectangular Polyethersulfone membranes of 0.45 μm pore size (Supor 450, Pall Corporation, Port Washington, N.Y.) grid side up on the surface of the bulbar conjunctiva and gentle pressure was applied. Membranes were peeled of the surface of the conjunctiva and suspended in 0.5 ml lysis buffer RLT (Qiagen, Valencia, Calif., USA) containing 1% 2-mercaptoethanol (SIGMA) and stored at −80° C. until total RNA was isolated from them.

Samples were obtained from 21 patients with tear dysfunction, including 12 patients with Sjögren's syndrome aqueous tear deficiency and 9 patients with non-Sjögren's syndrome tear dysfunction. Nine subjects served as normal controls with no irritation symptoms or ocular surface disease.

1. RNA Isolation and Reverse Transcription

Total RNA was isolated using a RNeasy Mini Kit (Qiagen) following the manufacturer protocol. Briefly, samples were heated at 37° C. for 10 min, vortexed, and passed through a QIAshredder column (Qiagen). Samples were applied to an RNeasy mini spin column and washed with two different buffers provided in the kit. The RNA was eluted by pipetting 50 μl of RNase-free water directly onto the center of the silica-gel membrane. The RNA concentration was measured by its absorption at 260 nm using a spectrophotometer (NanoDrop 2000, Thermo Scientific, Wilmington, Del., USA) and samples were stored at −80° C. until use in assays to measure gene expression.

First-strand cDNA was synthesized from 500 ng of total RNA with random hexamers using M-MuLV reverse transcriptase (Ready-To-Go You-Prime First-Strand Beads; Amersham Pharmacia Biotech, Inc., Piscataway, N.J.) as previously described.

2. Preparation of Gene Specific Standards

Template standard of known DNA sequence and concentration serves two main purposes. It functions as a positive control and as a reference for measuring the exact copy number of a transcript in an unknown sample.

Conventional Polymerase Chain Reaction (PCR) was performed to prepare template standards for absolute quantification of RNA obtained from patients and controls. cDNA was synthesized in the same manner as the patient and control samples from total human RNA (50 ng/ul, Applied Biosystem) and was used for PCR in a total volume of 50 μl containing the following: 10 μl of specific primers (Table 1) 0.4 mM of each dNTP, 1.5 mM of MgSO4, and 1 U of Taq polymerase (Invitrogen Carlsbad, Calif., USA). PCR was performed in a 2720 thermal cycler (Applied Biosystems) with parameters consisting of pre-denaturation at 94° C. for 2 min, followed by 40 cycles of denaturation at 94° C. for 1 min, annealing at 60° C. for 30 sec, and extension at 72° C. for 1 min. The final extension was conducted at 72° C. for 7 min.

TABLE 1
Primers used for exemplary conventional and real time PCR

| Gene | | Band Size | Catalog Number SA: |
|---|---|---|---|
| Symbol | Description | (bp) | SABiosciences |
| MMP3 | Matrix metallopeptidase 3 | 94 | SA: PPH00235E |
| MMP9 | Matrix metallopeptidase 9 | 63 | SA: PPH00152E |
| IFNγ | Interferon gamma | 129 | SA: PPH00380B |
| IL17A | Interleukin 17A | 154 | SA: PPH00537B |

After amplification, the PCR mixture (10 μL) was separated by electrophoresis on a 1.5% agarose gel, stained with ethidium bromide and a digital image of the gel was acquired (Image Station Model 2000R; Eastman Kodak, New Haven, Conn., USA) before and after purification using a PCR Purification Kit following the manufacturer protocol (Spin-50 mini-column, USA Scientific, Inc, Ocala, Fla., USA). Only the products that amplify a single and distinct band were used for preparing the standards.

The DNA concentration was measured by its absorption at 260 nm using a spectrophotometer (NanoDrop 2000, Thermo Scientific). The concentration is converted from ng per μl to copy number per μl using the following formula: (C×10⁻⁹/MW)×NA (C: template concentration ng/μl, MW: template molecular weight in Daltons, NA: Avogadro's constant, 6.022×10²³). Serial 10 fold dilutions were prepared from each DNA template starting from 10⁹ to 10.

3. Absolute Real Time Polymerase Chain Reaction (Rt-PCR)

Absolute rt-PCR was performed using SYBR® Green reagents. SYBR® Green reagents use a SYBR® Green I dye, a double-stranded DNA binding dye, to detect PCR products as they accumulate during PCR cycles. This dye binds nonspecifically to all double-stranded DNA sequences, so to avoid false positive signals; the user has to check for nonspecific product formation using melt curve on gel analysis.

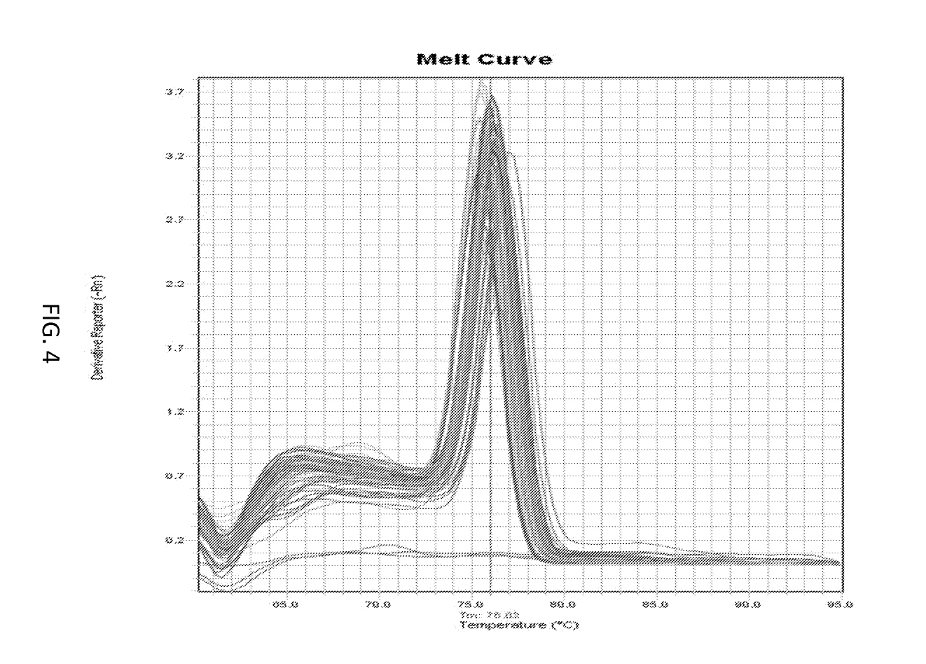

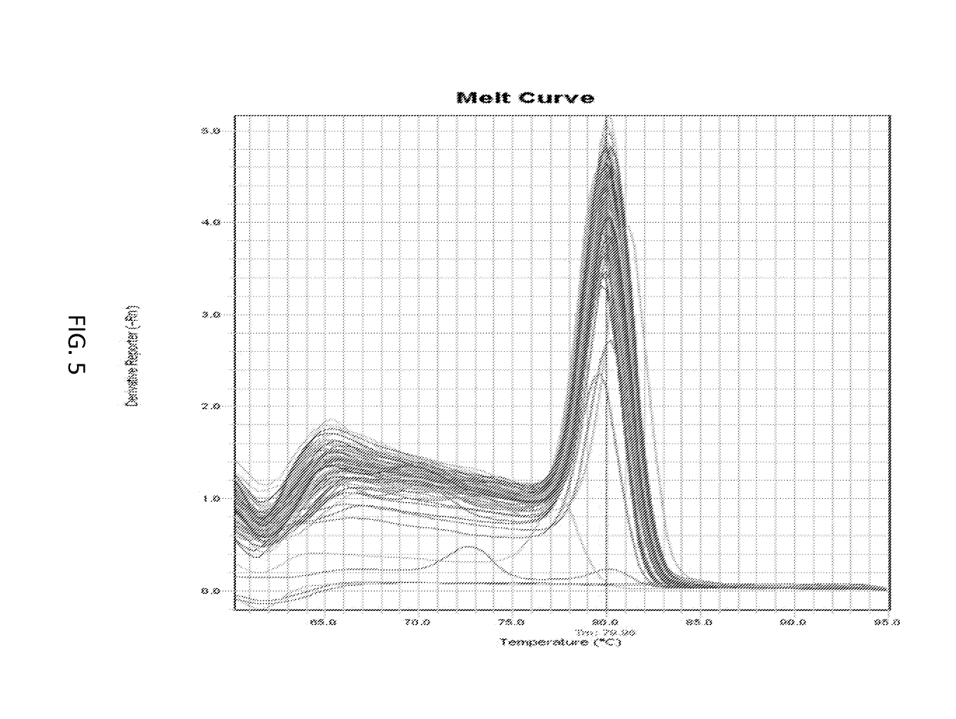

cDNA aliquot (3 μl) from samples (controls and patients) and standards was used for Absolute rt-PCR in a total volume of 10 μl containing the following per reaction: 0.2 μl of the specific primers used in the conventional PCR (Table 1), 5 μl of 2×SYBR® Green PCR Master Mix (Applied Biosystems). Absolute rt-PCR was performed in a StepOnePlus™ Real-Time PCR System (Applied Biosystems) with parameters consisting of pre-denaturation at 95° C. for 22 sec, followed by 40 cycles of denaturation at 95° C. for 1 sec, annealing and extension at 60° C. for 1 min. After this a cycle of denaturation was performed to generate a specific melt curve for each gene (examples are shown in FIGS. 2-5).

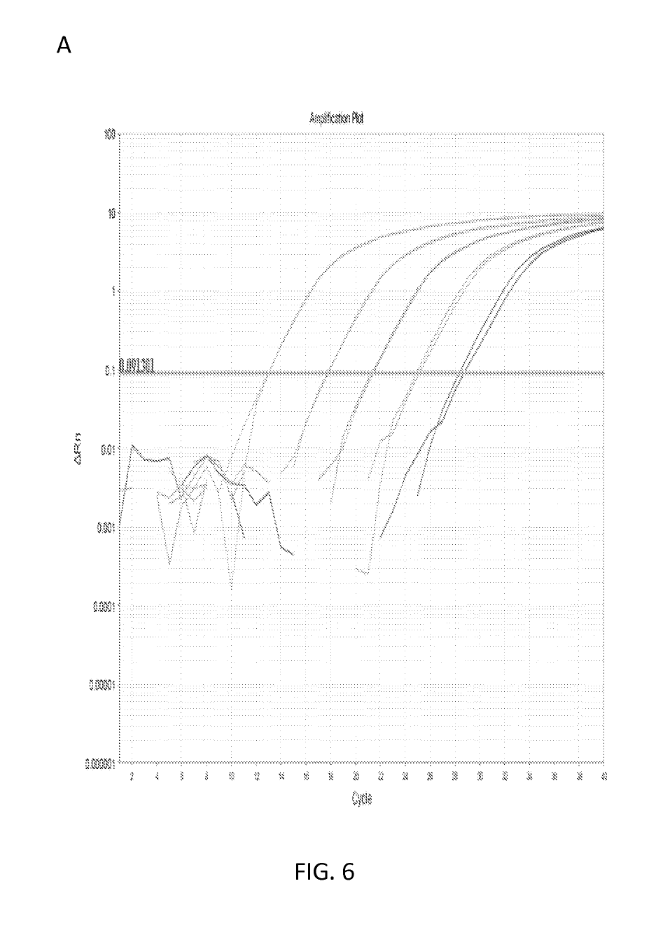

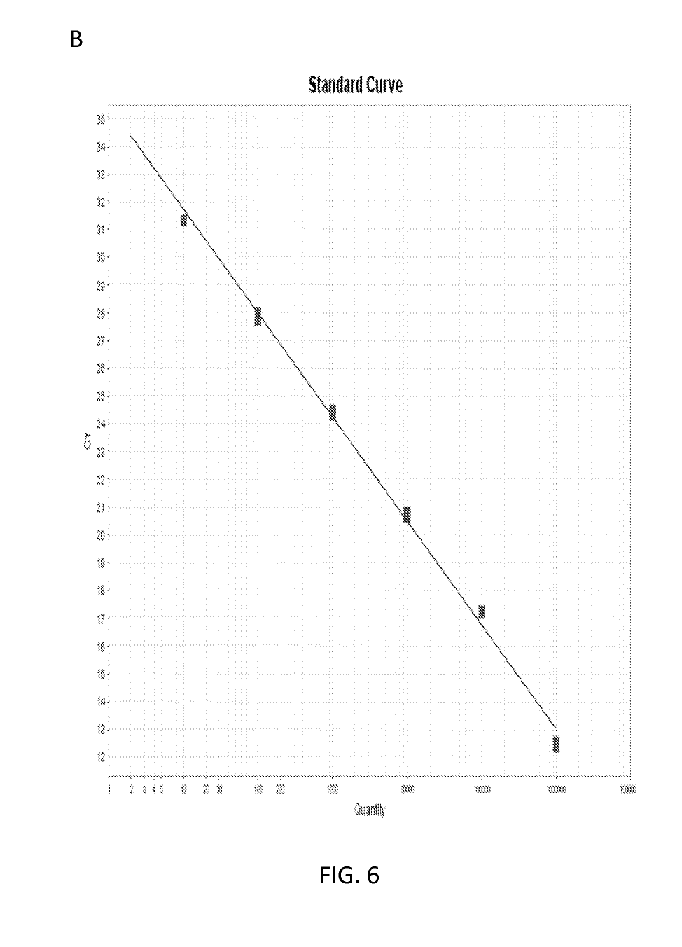

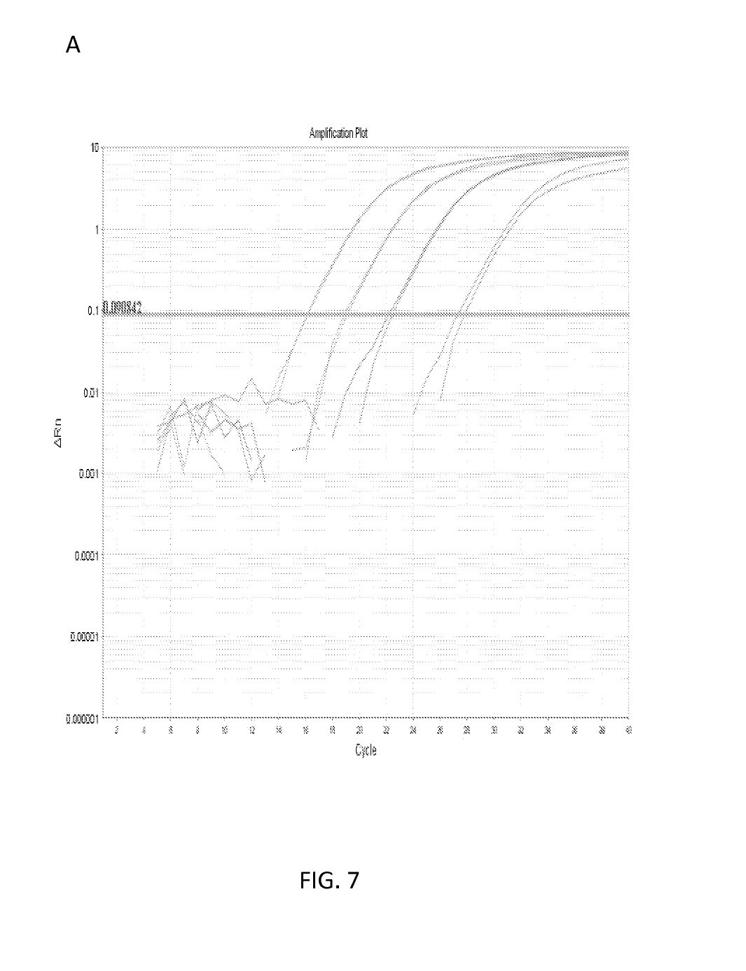

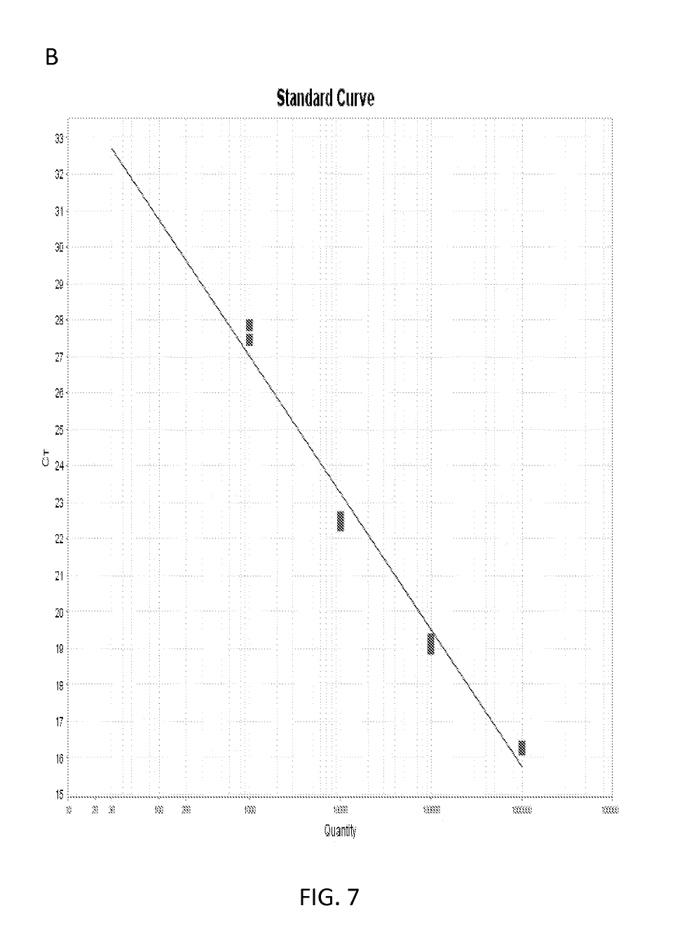

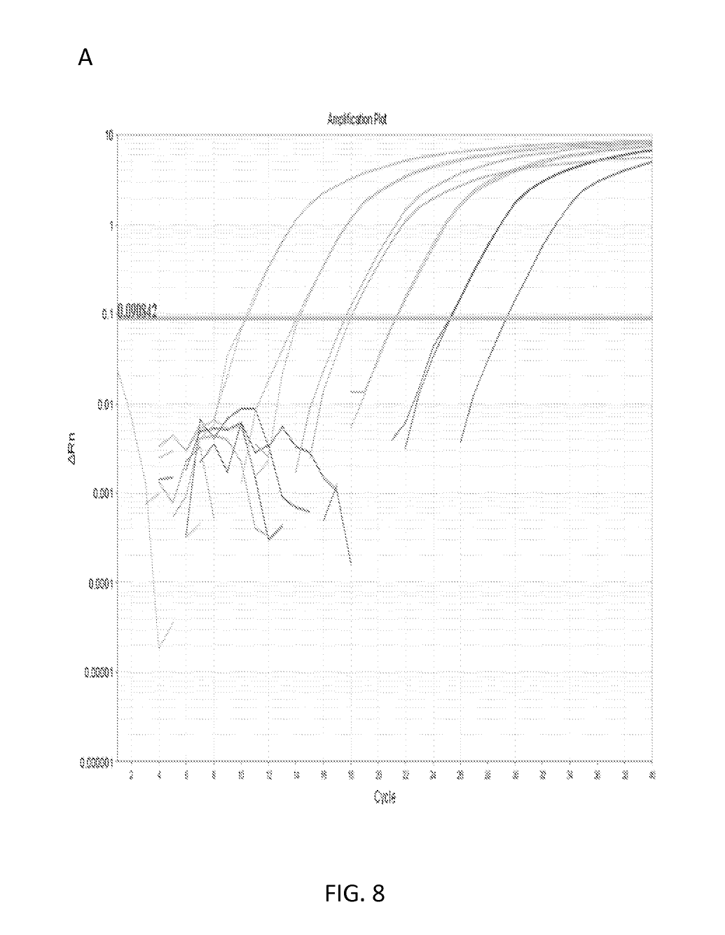

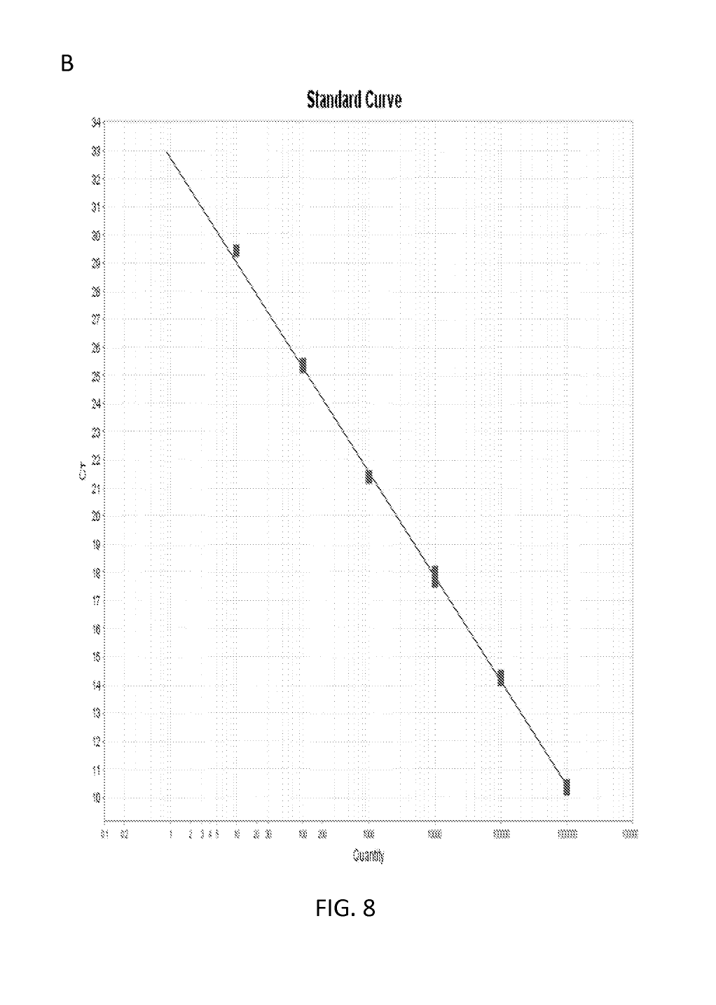

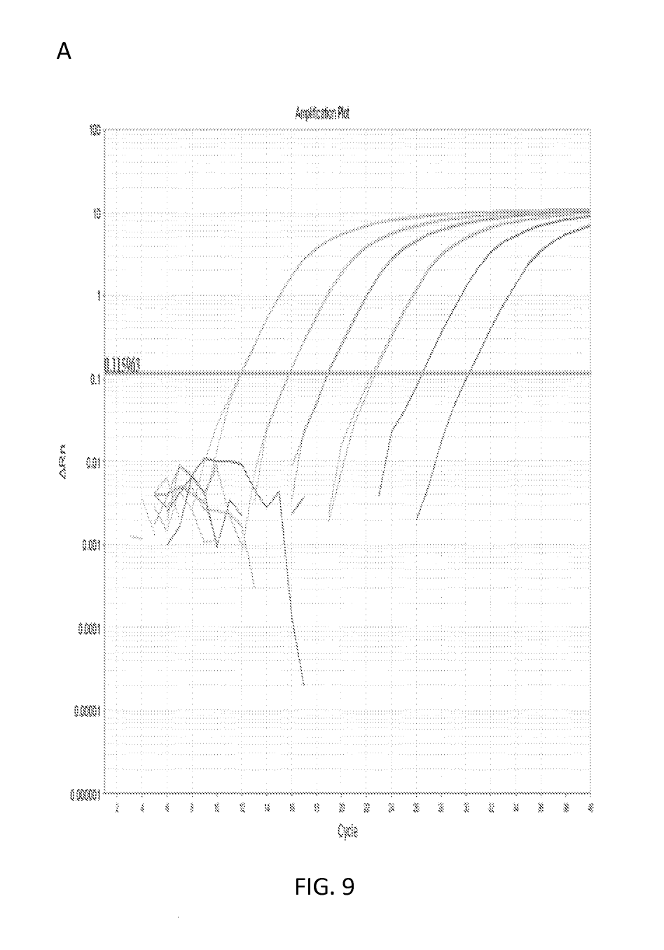

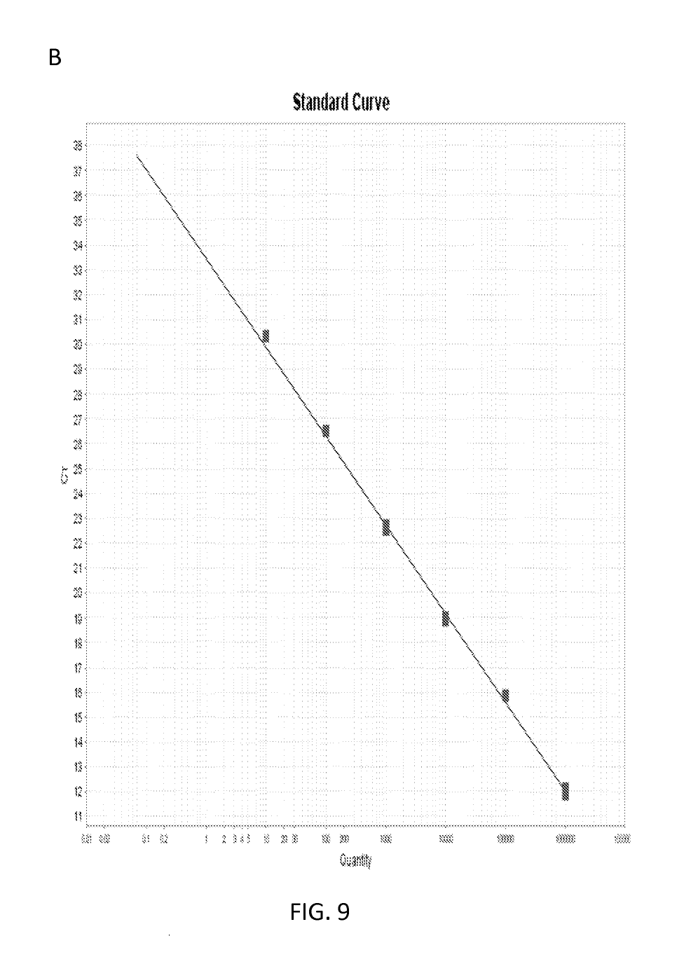

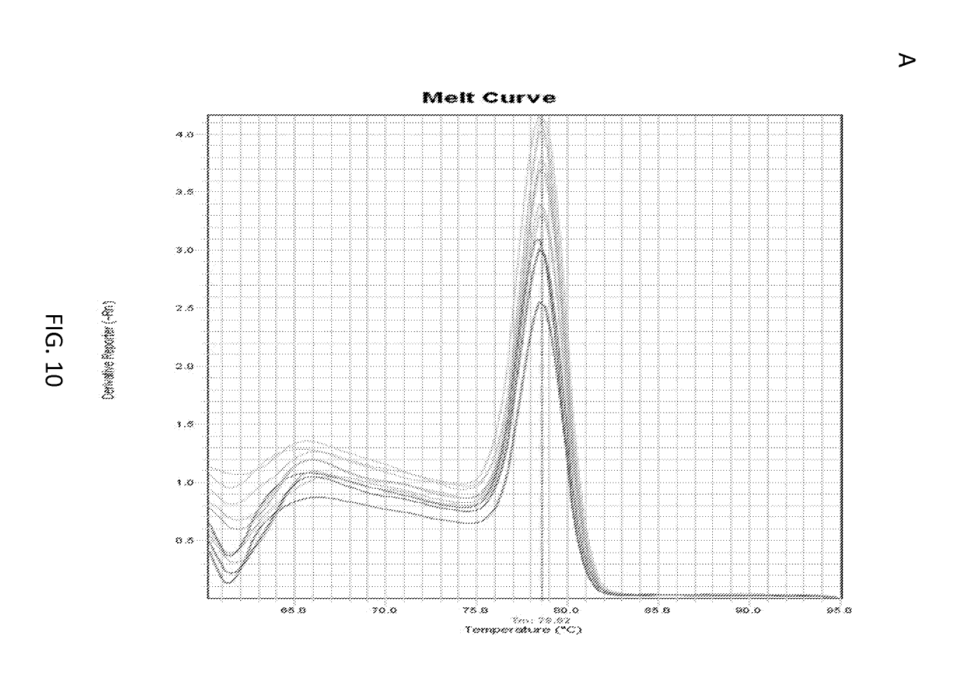

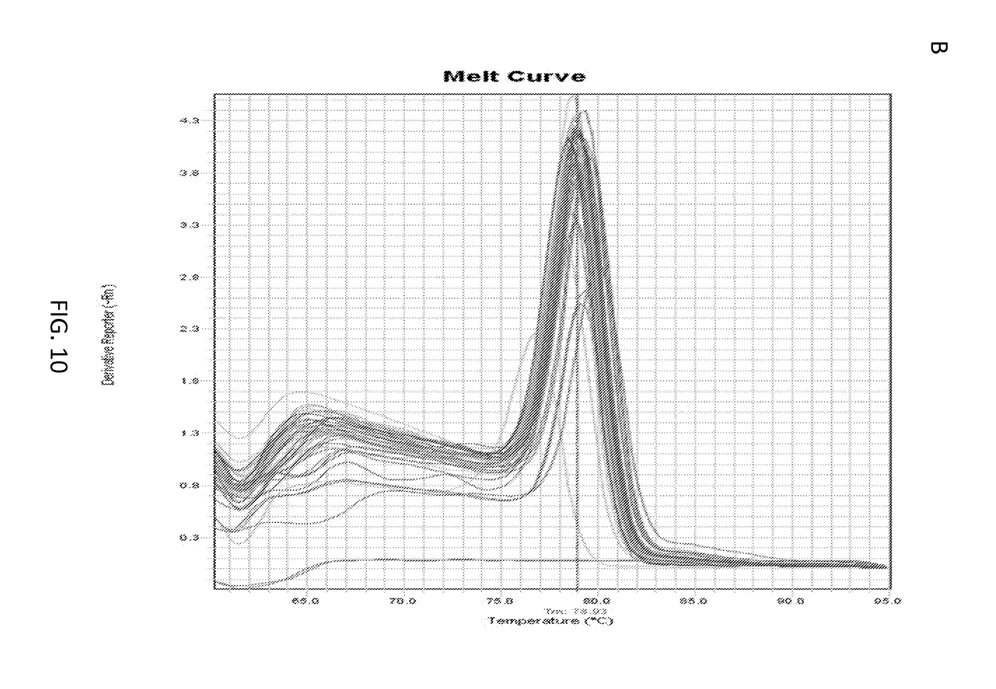

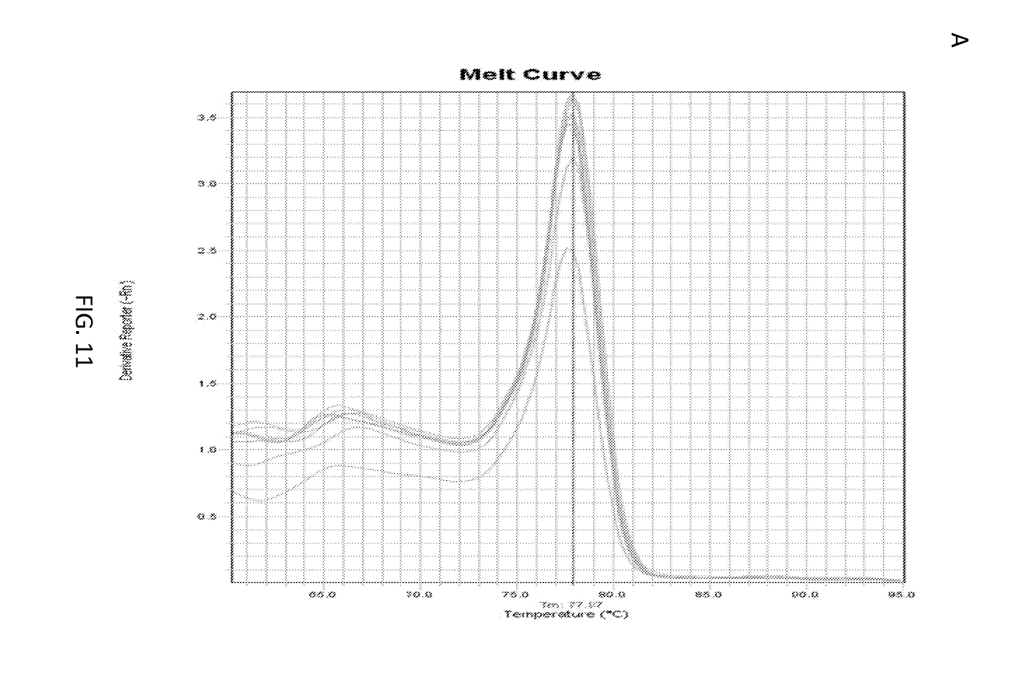

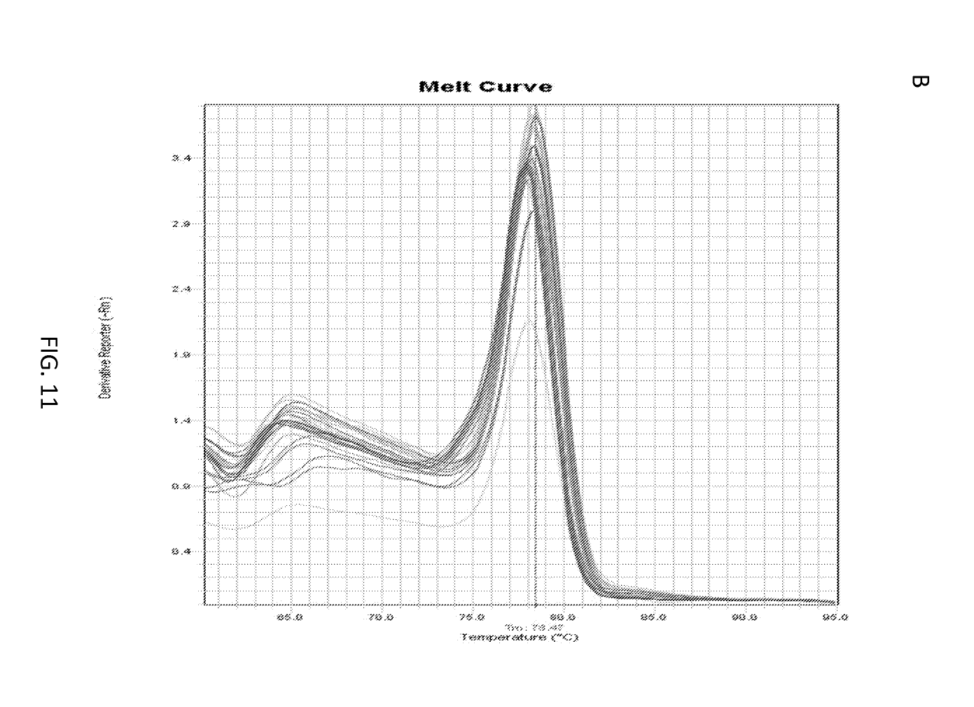

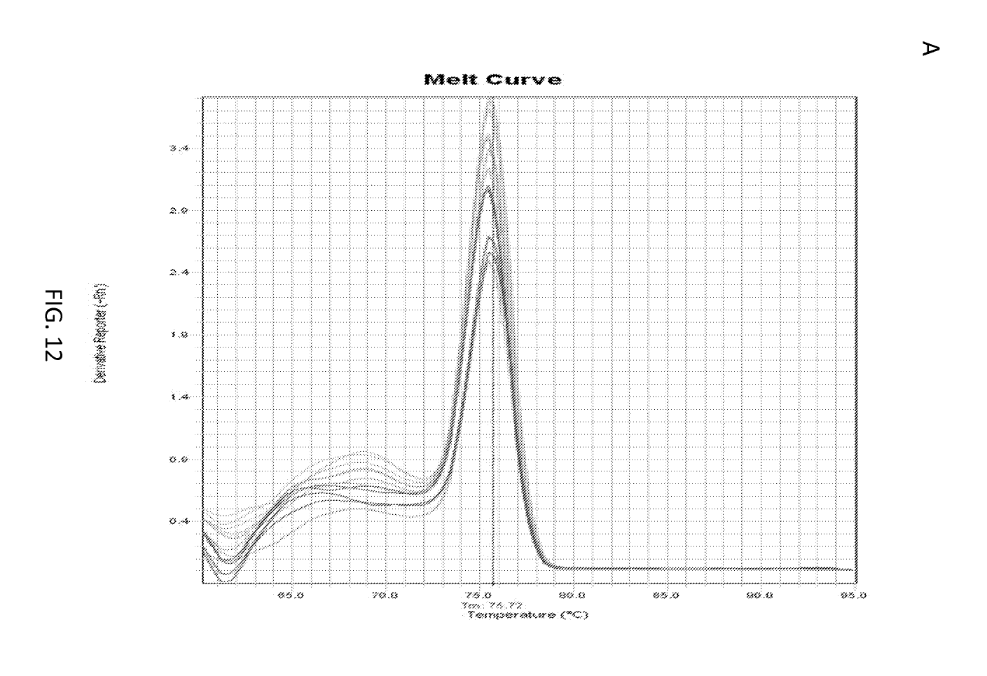

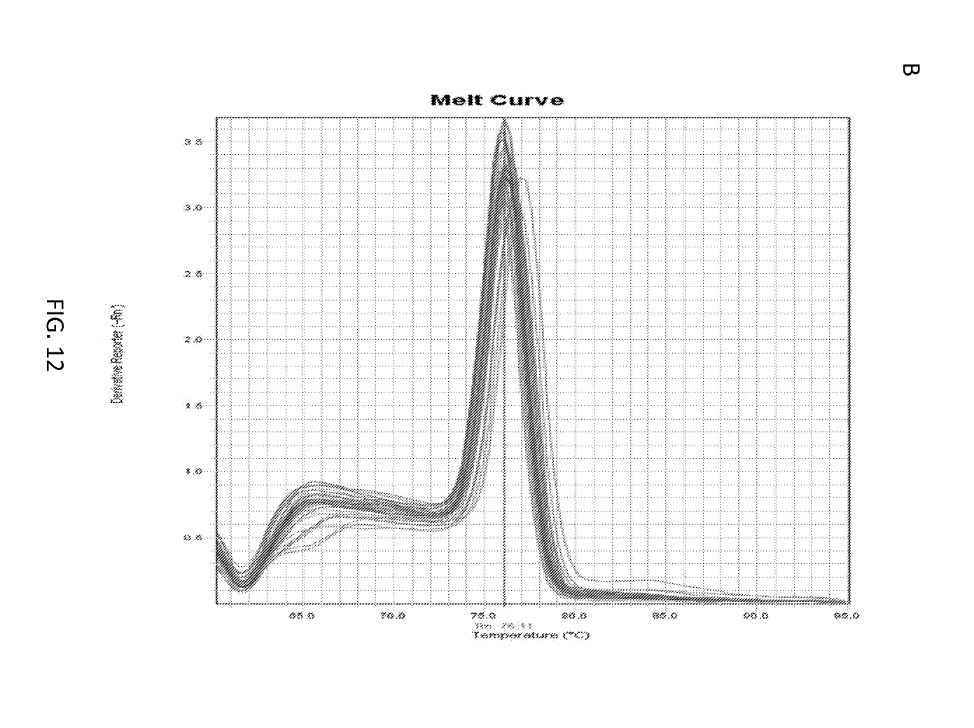

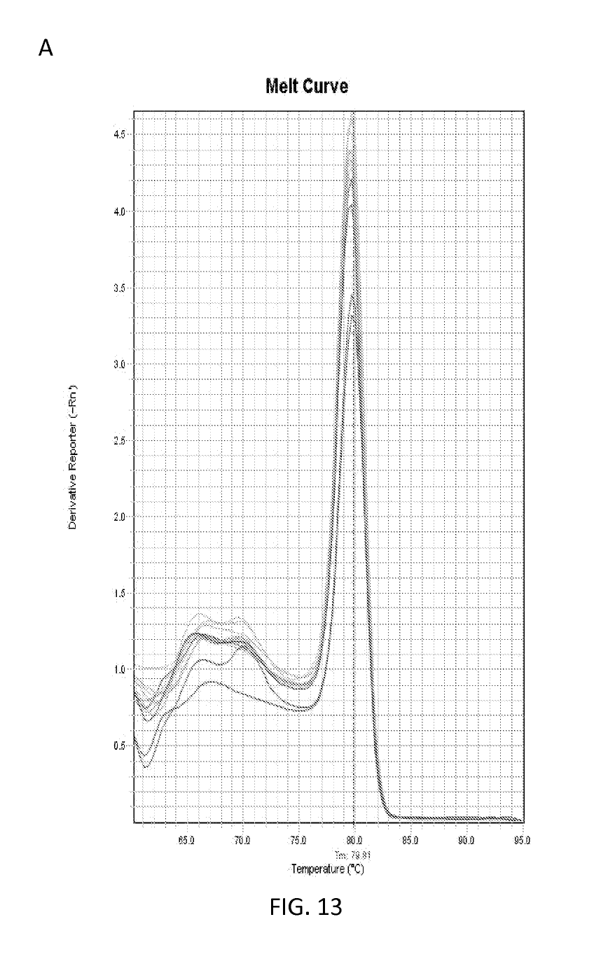

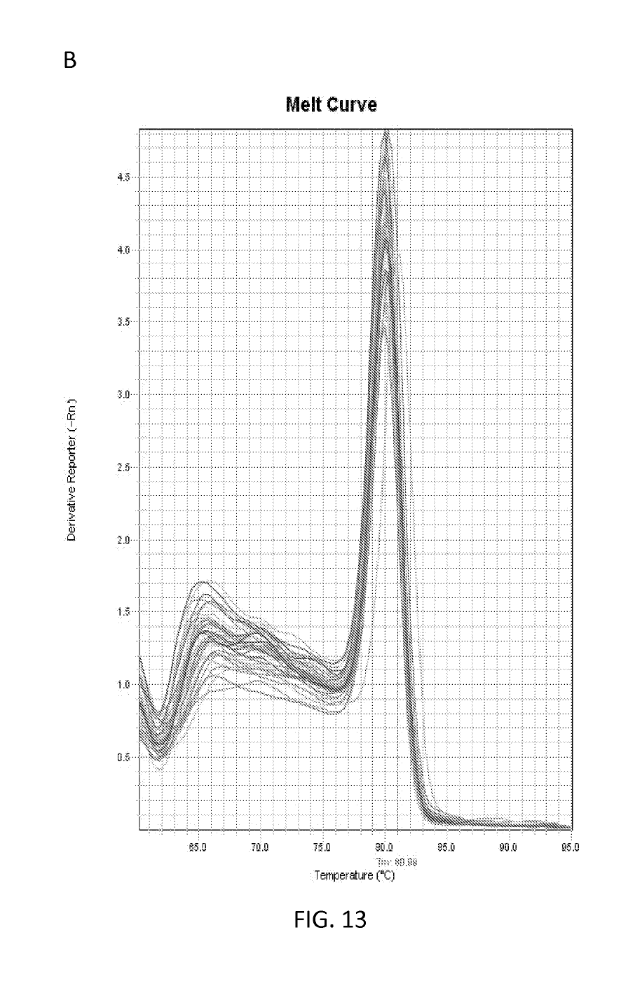

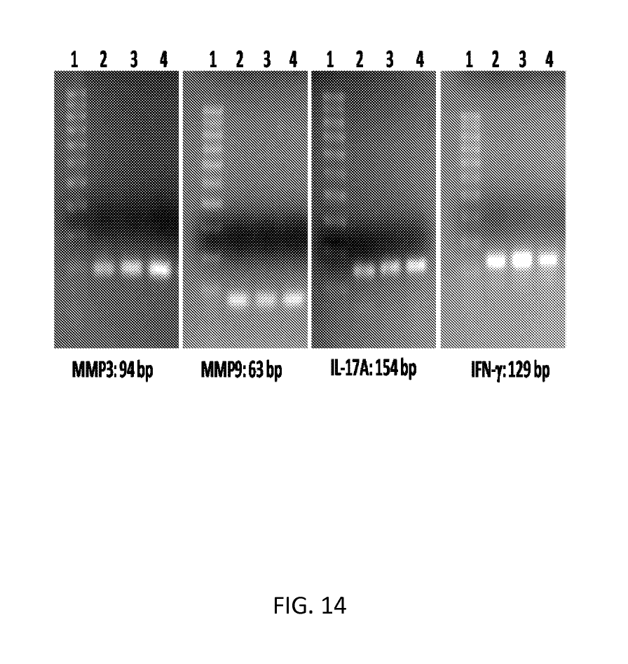

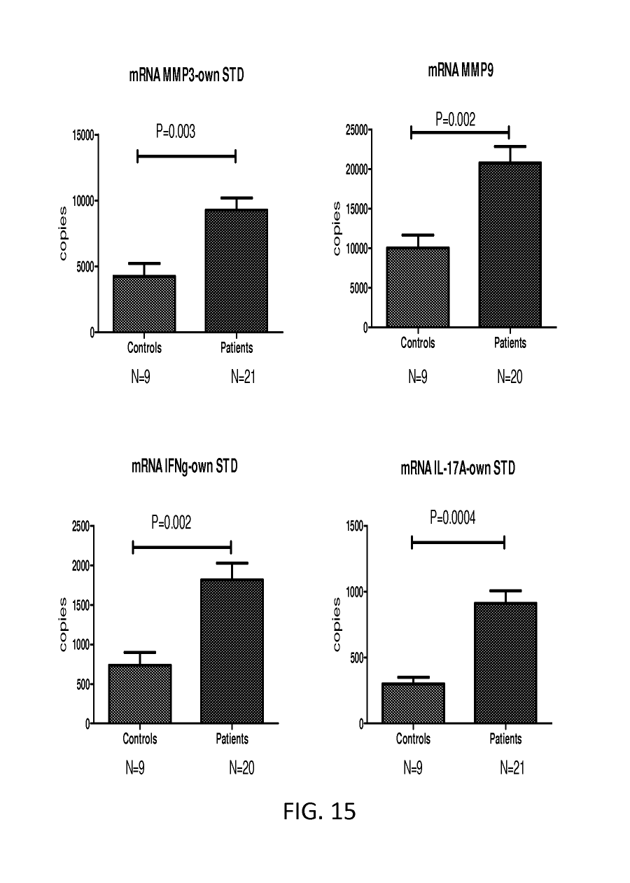

Samples and standards were assayed in duplicate. A nontemplate control and total RNA without retrotranscription were included in all the experiments to evaluate PCR specificity and DNA contamination of the reagents used. The Standard Curve screen displays the standard curve for samples designated as standards for each gene (FIGS. 6-9). ΔRn is the magnitude of normalized fluorescence signal generated by the SYBR Green at each cycle during the PCR amplification The threshold cycle (C_{T}) is the PCR cycle number at which the fluorescence level meets the threshold. The same melt temperature for PCR products amplified from those standards and controls and patients samples were confirmed (FIGS. 10-13). The calculation of the copy number of the genes of interest in patients and controls was calculated from those standard curves.

After analysis the real time PCR products for all the genes, were pooled in different tubes, purified using a PCR Purification Kit Protocol (Spin-50 mini-column, USA Scientific, Inc) and run on an agarose gel for verifying a single amplification product (one band) of the appropriate size.

Tube 1: All standards

Tube 2: All normal controls

Tube 3: All patients

The size and identity of the PCR products was verified by cloning each into a sequencing vector using a TOPO TA Cloning® Kit for Sequencing (Invitrogen), following the manufacturing protocol. The sequences were verified using the BLAST program.

Statistical Analysis

The calculation of the copy number of the genes of interest in patients and controls samples was calculated from standard curves. Samples obtained from Sjögren's Syndrome and non-Sjögren's Syndrome tear deficiency were combined (n=21) and compared to normal controls.

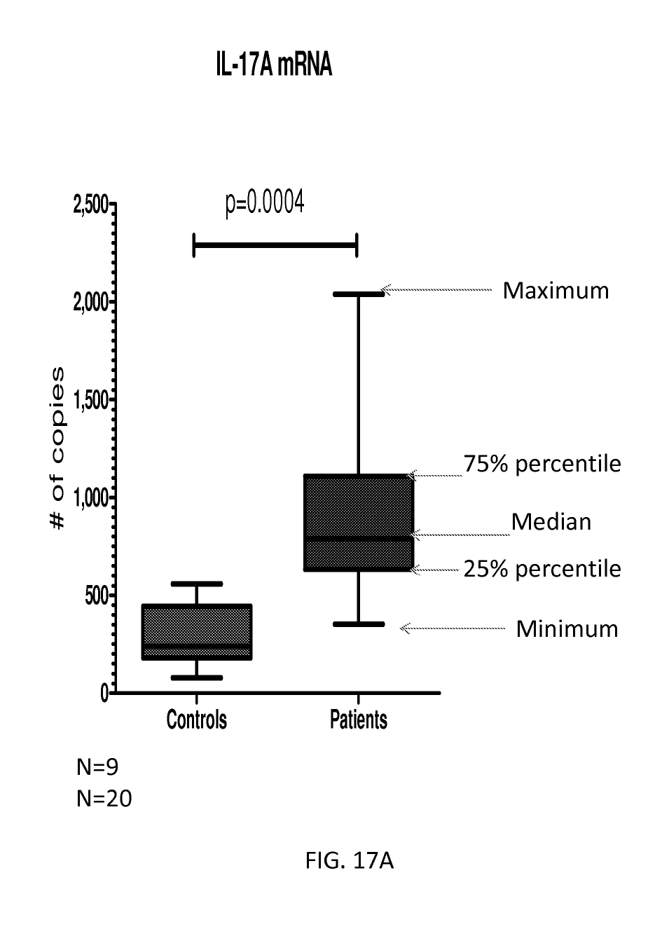

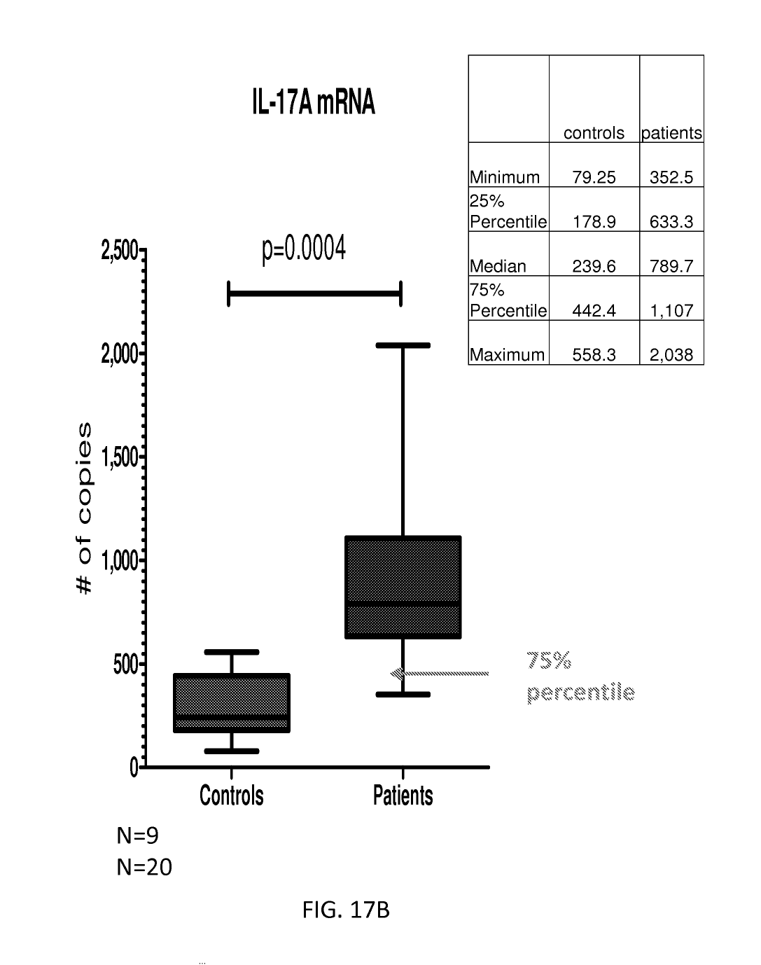

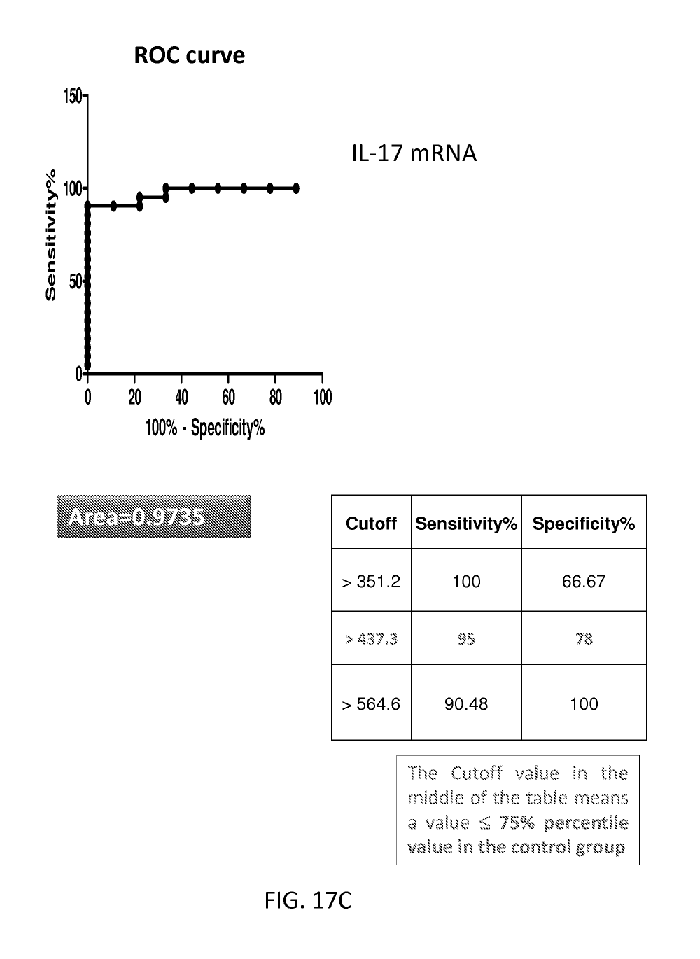

Results are presented as the median with boxes show the 25 and 75% percentiles. (FIGS. 17A, 17B, 18A, 19A, 20A, 21A and 22A). Statistical differences were evaluated by the Student's t-test for independent samples. Values of p≦0.05 were considered significant. Statistical tests were performed using GraphPad Prism 5.02 software (GraphPad Software Incorporation, San Diego, Calif., USA).

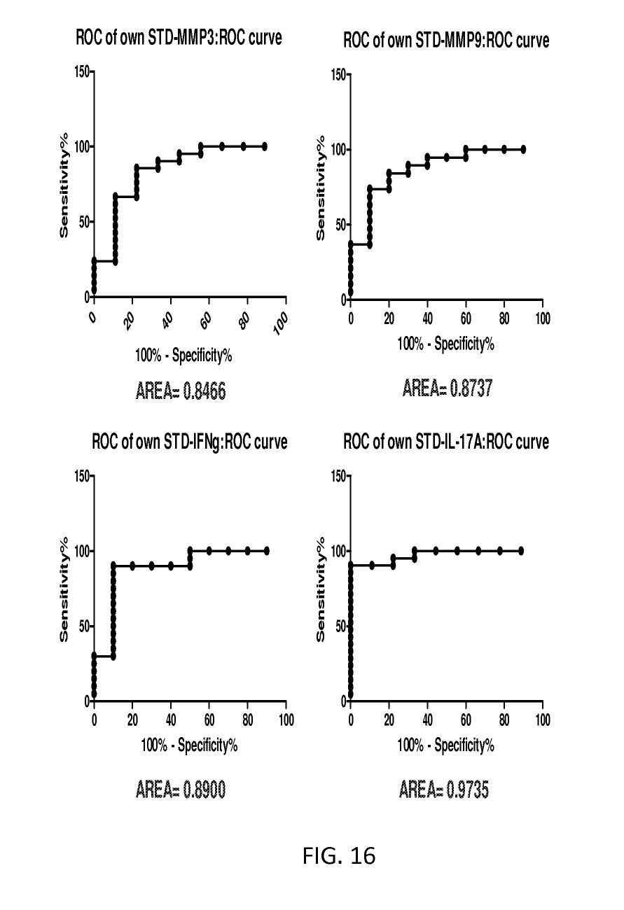

The sensitivity and specificity was analyzed, ROC curves were generated (FIG. 16) and the areas under ROC curves were calculated. The area under a ROC curve quantifies the overall ability of the test to discriminate between those individuals with the disease and those without the disease. A truly useless test (one no better at identifying true positives than flipping a coin) has an area of 0.5. A perfect test (one that has zero false positives and zero false negatives) has an area of 1.00. (FIGS. 17C, 18B, 19B, 20B, 21B and 22B).

TABLE 2
Summary of results from samples obtained from
patients with tear dysfunction (Sjögren's syndrome
and non- Sjögren's syndrome) and normal controls.

| | MMP-3 | | MMP-9 | | IFN g | | IL-17A | |
|---|---|---|---|---|---|---|---|---|
| | controls | patients | controls | patients | controls | patients | controls | patients |
| Mean | 4259 | 9258 | 10048 | 20783 | 737.3 | 1817 | 299.8 | 911 |
| Std. Deviation | 2912 | 4227 | 5084 | 9132 | 512.8 | 930.6 | 157.6 | 439.3 |
| Std. Error | 970.5 | 922.4 | 1608 | 2095 | 162.1 | 208.1 | 52.54 | 95.85 |
| Lower 95% CI of mean | 2021 | 7334 | 6411 | 16381 | 370.5 | 1382 | 178.6 | 711.1 |
| Upper 95% CI of mean | 6497 | 11182 | 13685 | 25184 | 1104 | 2253 | 420.9 | 1111 |

| | | | | |
|---|---|---|---|---|
| p value | 0.0032 | 0.002 | 0.002 | 0.0004 |

### Example 2

### Exemplary Clinical Embodiments

In embodiments of the invention, an individual with one or more symptoms of ocular surface inflammation is in need of treatment thereof. As part of the treatment determination, the individual may first be subjected to exemplary methods of the invention wherein expression of one or more particular genes is identified, and such analysis dictates the suitable treatment or at the very least narrows the selection of suitable treatments.

Methods to identify particular gene expression levels are known and routine to those of skill in the art. One or more samples from the particular individual may be obtained as part of the method, or the sample(s) may be obtained by a party other than the party that performs the expression analysis. Exemplary materials to be used may include one or more of subject labels; container bags (for example, plastic); tissue collection tubes that may be pre-filled with storage media; EyePrim® device (Opia Technologies, Paris, France); sterile tweezers (such as plastic) for handling membrane; cold bricks—keep them at −20° C. after arriving; insulated shipping kit; and shipping labels.

Nine healthy subjects were employed in the study, in which their Tear Break-up time (TBUT) was greater than 8 seconds. They had no corneal fluorescein staining or conjunctival lissamine green staining. Their Ocular Surface Disease Index (OSDI) symptom severity Score was less than 20. Twenty dry eye (DE) patients [9 non Sjögren Syndrome and 11 with Sjögren Syndrome (dry mouth and serum autoantibody +)] were used as experimental subjects. Their tear break-up time was less than 7 seconds; the corneal fluorescein staining had a range of 1-24; their conjunctival lissamine green staining had a range of 1-24; and their OSDI score was greater than 22 (range of 23-38).

In exemplary methods, samples were obtained from the individuals and were processed for RNA isolation (including tissue dissociation, cell lysis, RNA binding to a membrane, washing of the membrane, and elution); quantification of RNA, such as with a spectrophotometer; cDNA synthesis and real time PCR, such as with specific primers and a PCR master mix.

Exemplary real time PCR methods were employed. Real-time PCR performed using specific primers for IL-17A, IL-6, MMP-3, MMP-9, IFN-γ, HLRA-DRA and/or SPRR-1A. The detection system was a SYBR® Green I detection system. Controls were employed to test for contamination, including no-template control and RNA. A dissociation curve was utilized to verify the identity of each gene amplification product. Results were analyzed by absolute method using standard curves. The student t-test for independent samples was used for statistical analysis between control and patients samples. The receiver operator characteristic (ROC) analysis was performed to characterize the diagnostic test and select cutoff values, for example.

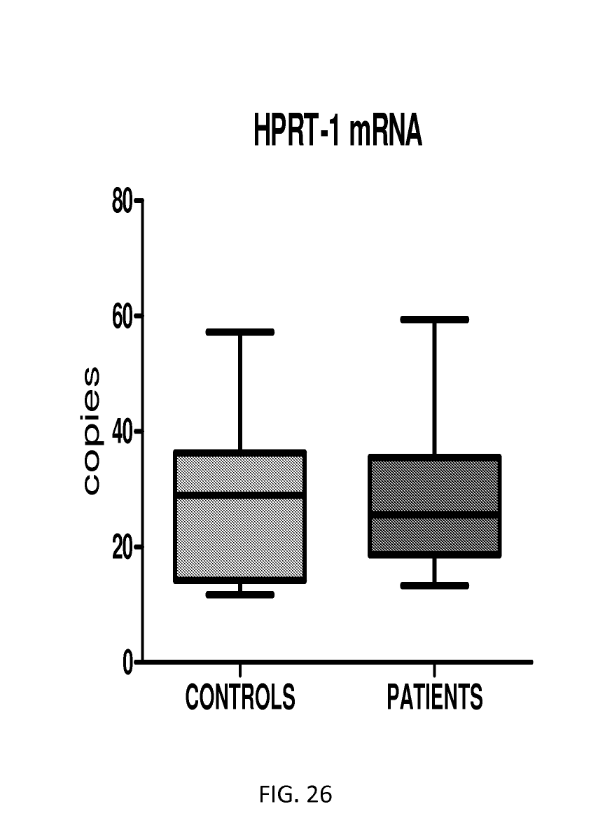

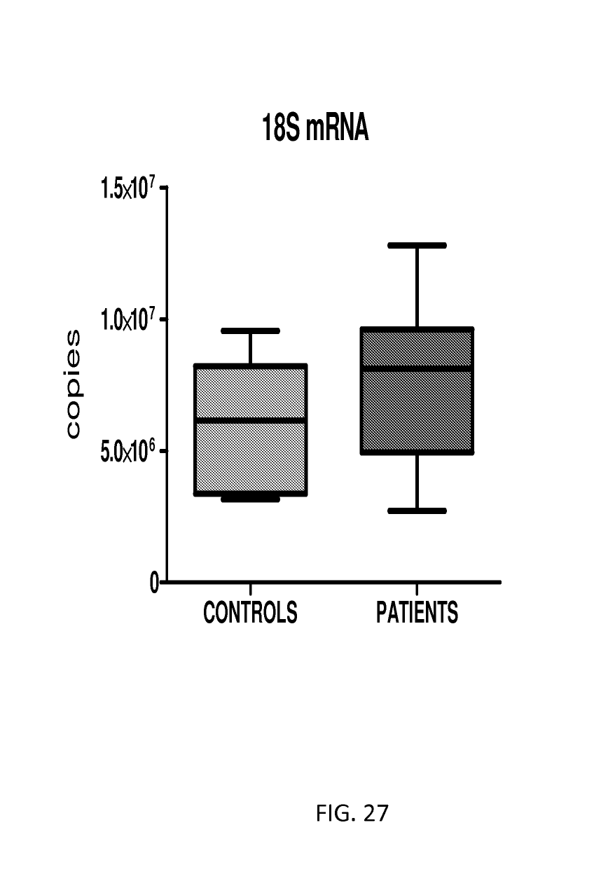

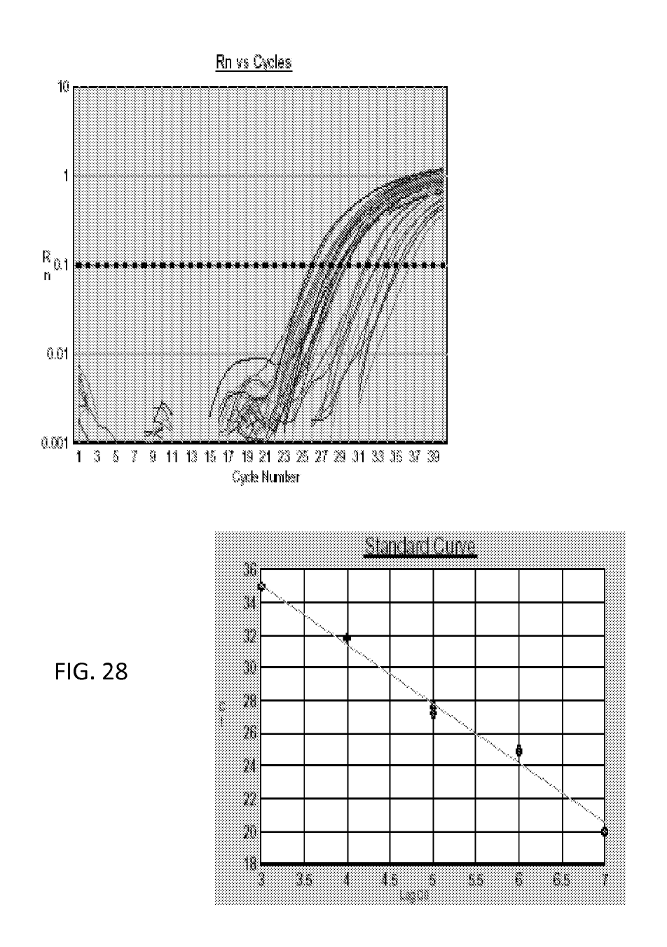

FIG. 28 shows an exemplary real time PCR amplification plot and the standard curve. The amplification plot represents Rn (The magnitude of normalized fluorescence signal generated by the SYBR Green a each cycle during the PCR amplification) vs. Cycle. The standard curve plot represents number of threshold cycle (CT) vs the quantity (log) for samples designated as standards (from 10⁷ to 10³).

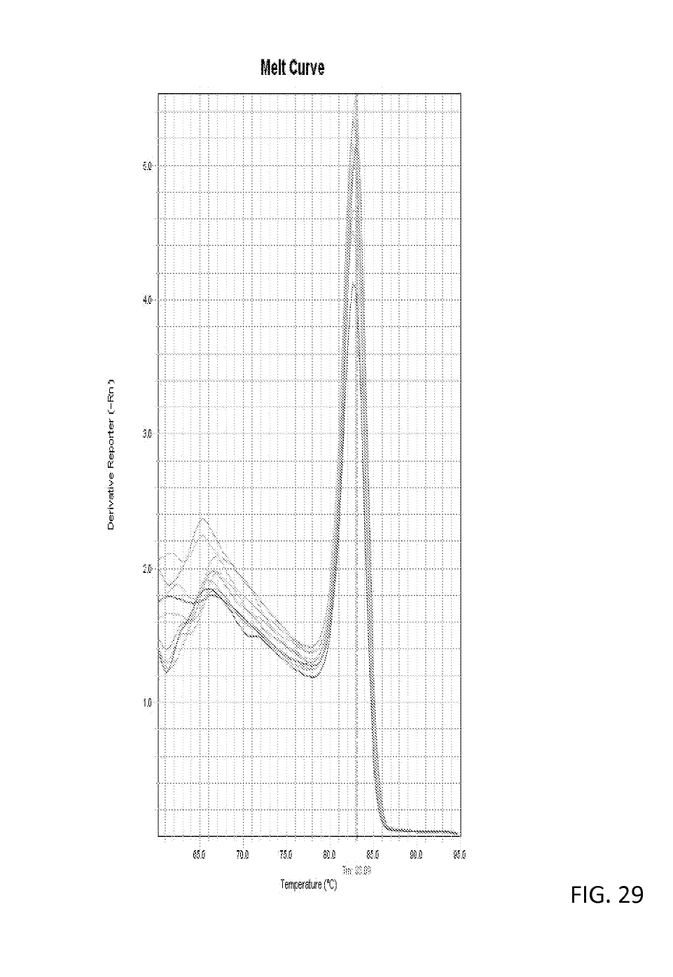

FIG. 29 illustrates an exemplary melt curve to verify the identity of each gene amplification product. Absolute RT-PCR was performed using SYBR® Green reagents. SYBR® Green reagents use a SYBR® Green I dye, a double-stranded DNA binding dye, to detect PCR products as they accumulate during PCR cycles. This dye binds nonspecifically to all double-stranded DNA sequences; to avoid false positive signals; the user checks for nonspecific product formation using melt curve.

Gene-specific qPCR template standards were employed. A known qPCR template standard serves two main purposes: it functions as a positive control and as a reference for measuring the exact copy number of a transcript in an unknown sample. There are several ways to generate a collection of full-length human and mouse cDNA constructs that are ideal PCR templates:

a) using high purity ion-exchange columns to purify the cDNA, linearized by a restriction enzyme and quantified to be diluted.

b) the target of interest can be amplified and either cloned or the PCR product can be purified and serially diluted.

c) long oligos or genes, can be purchased from a company and will provide a known amount of product.

d) serial cDNA dilutions can be made using RNA from a tissue or cell line with high levels of expression of the target of interest.

Standards were generated from commercial total RNA in which cDNA was synthesized, the targets of interest were amplified by PCR, and the PCR products were purified, quantified, and serially diluted.

Quantification of gene expression analysis is performed in embodiments of the invention. In specific embodiments, the gene expression analysis quantification encompasses determination of copy number for one or more particular genes. In specific embodiments, if the value of the copy number for expression of a particular gene from a sample of an individual is above the 75th percentile value of a control group, treatment of the disease is indicated and if untreated the individual is at risk for developing the disease or is at risk for increasing the severity of one or more symptoms of the disease.

In specific embodiments of the invention, the expression of one or more particular genes selected from the group consisting of MMP3, MMP9, IFNγ, IL17A, IL-6, HLD-DRA, MUC5AC, K7, IL-13, and a combination thereof is performed. FIGS. 17A and 17B show exemplary diagnostic results for IL-17A mRNA using nine controls and 20 patients. FIG. 17C shows an exemplary ROC curve for IL-17A mRNA studies. The area under a ROC curve quantifies the overall ability of the test to discriminate between those individuals with the disease and those without the disease. An uninformative test (one no better at identifying true positives than flipping a coin, for example) has an area of 0.5. A perfect test (one that has zero false positives and zero false negatives) has an area of 1.00. In embodiments of the invention, the methods of the invention encompass suitable sensitivity and specificity. Sensitivity may be considered the fraction of people with the disease that the test correctly identifies as positive. Specificity may be considered the fraction of people without the disease that the test correctly identifies as negative.

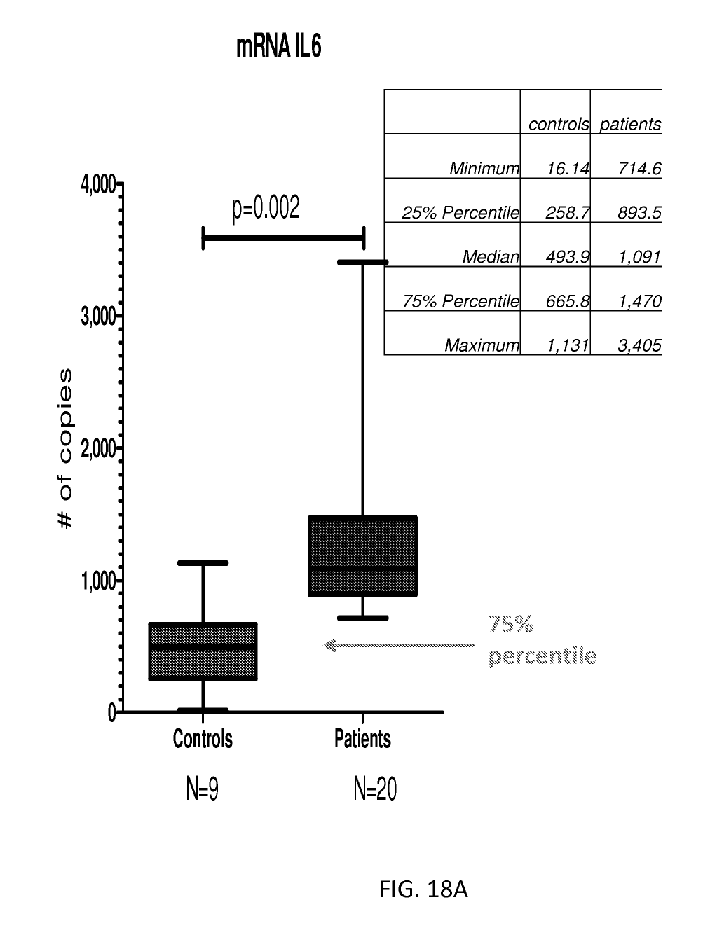

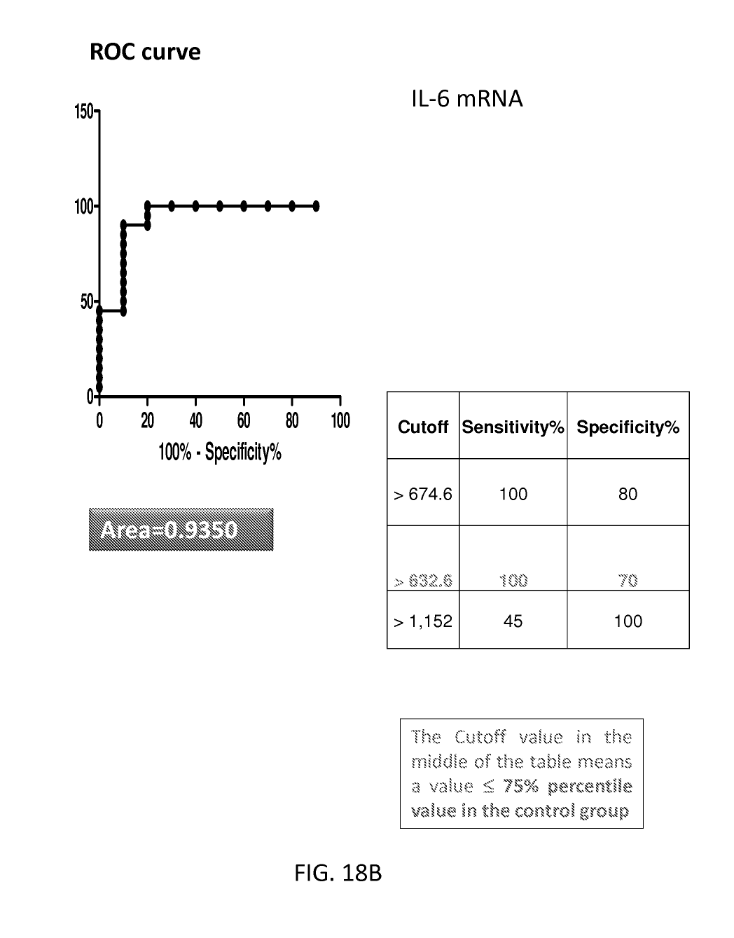

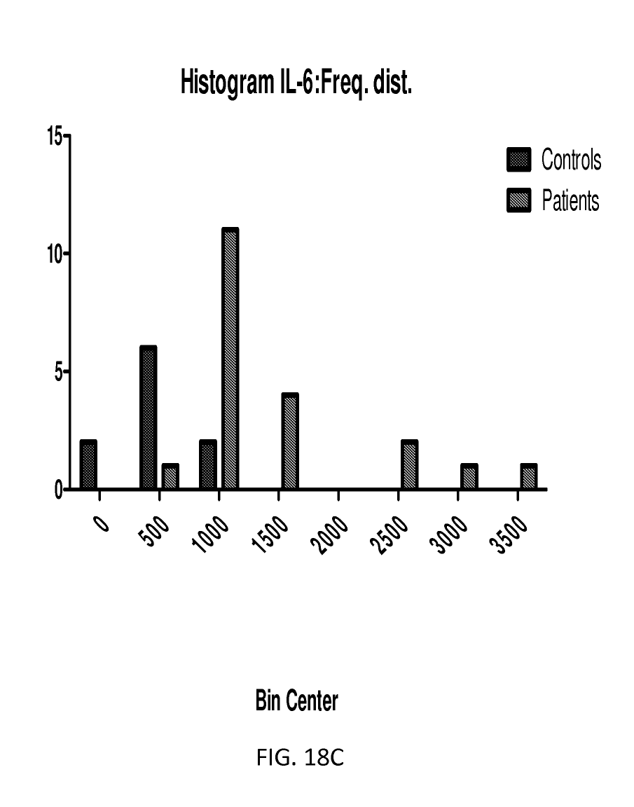

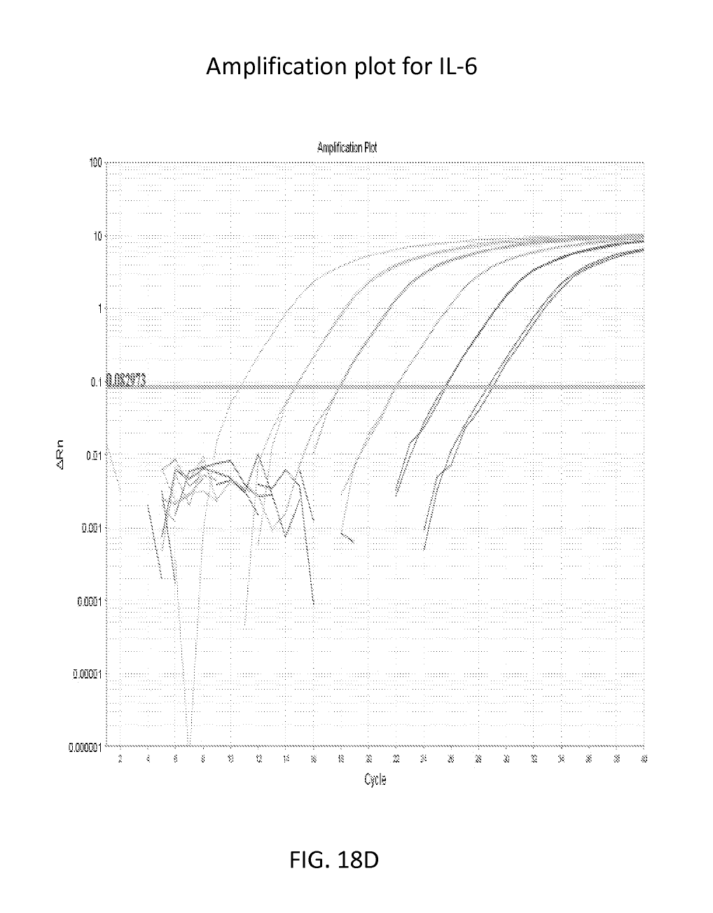

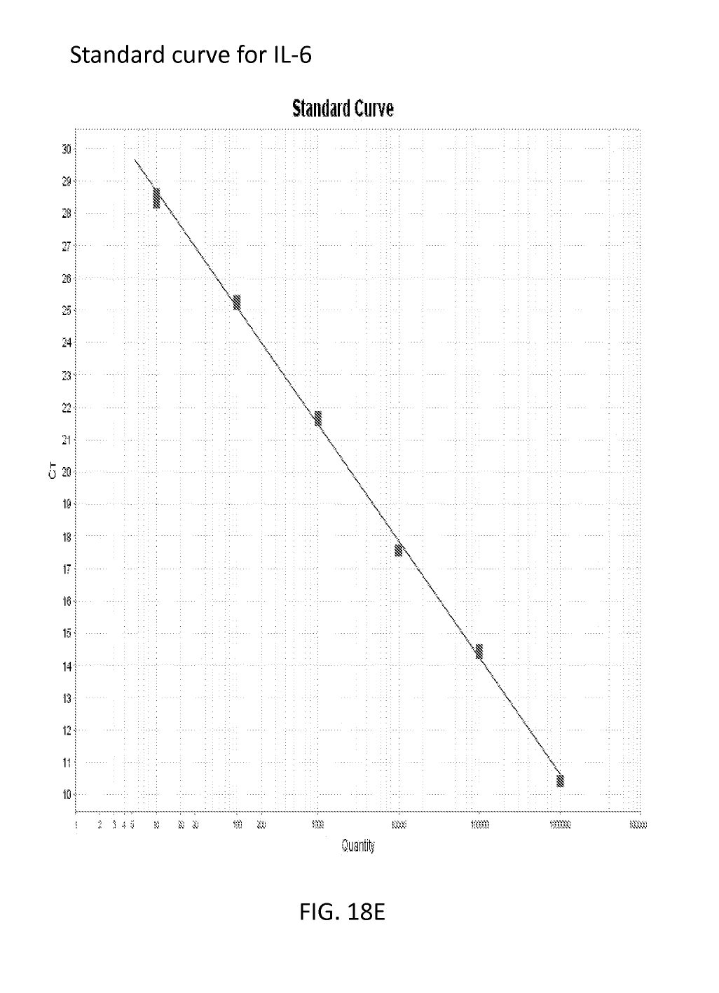

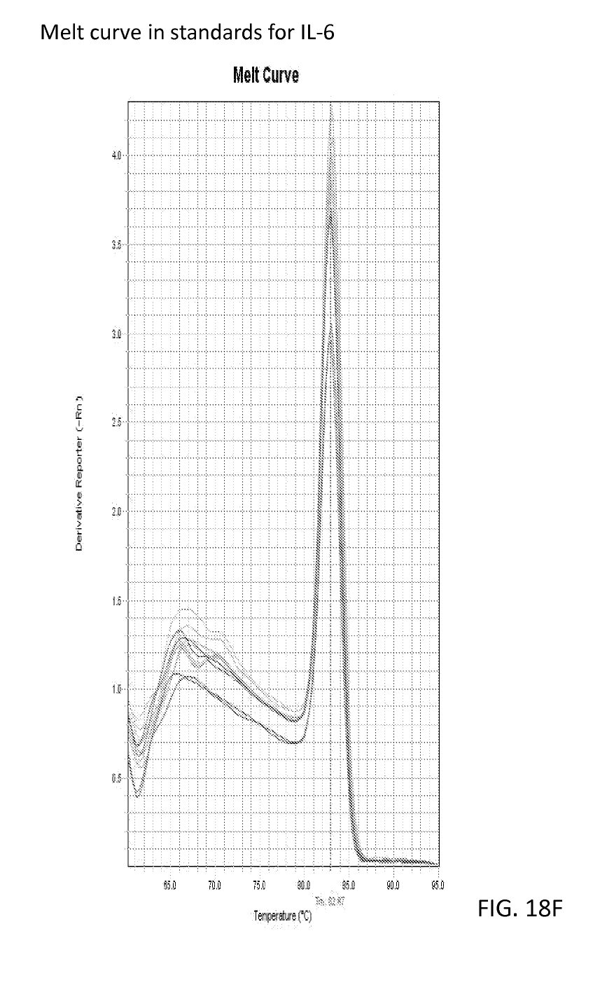

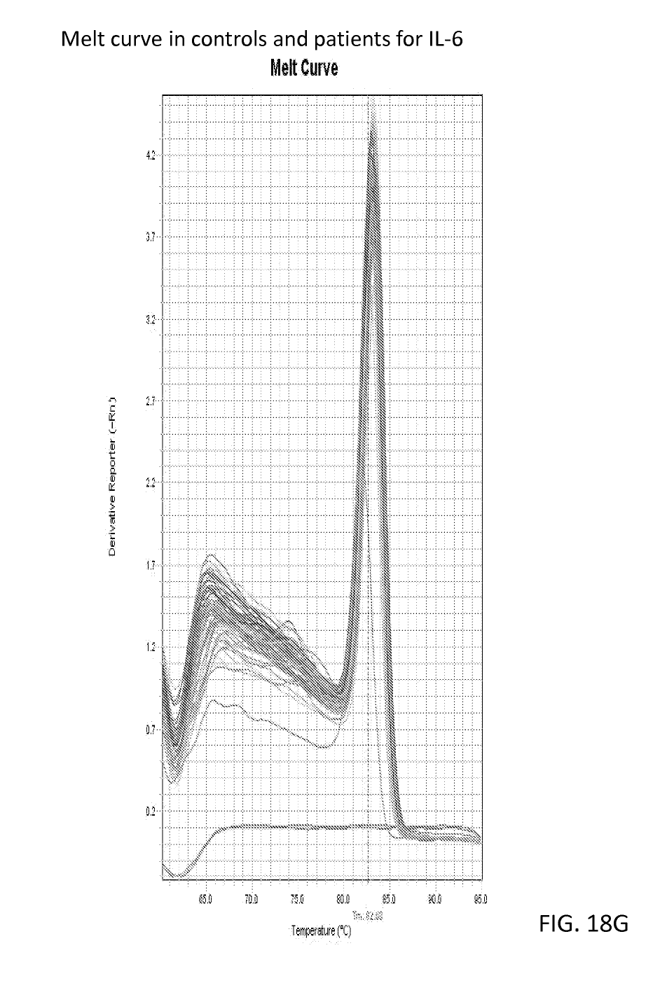

FIG. 18A shows diagnostic results for IL-6 mRNA. FIG. 18B shows an exemplary ROC curve for IL-6 mRNA. FIG. 18C shows exemplary diagnostic results as a histogram of the frequency distribution of IL-6 mRNA. FIG. 18D demonstrates an exemplary amplification plot for IL-6. FIG. 18E shows an exemplary standard curve for IL-6. FIG. 18F shows an exemplary melt curve in standards for IL-6. FIG. 18G demonstrates an exemplary melt curve in controls and patients for IL-6.

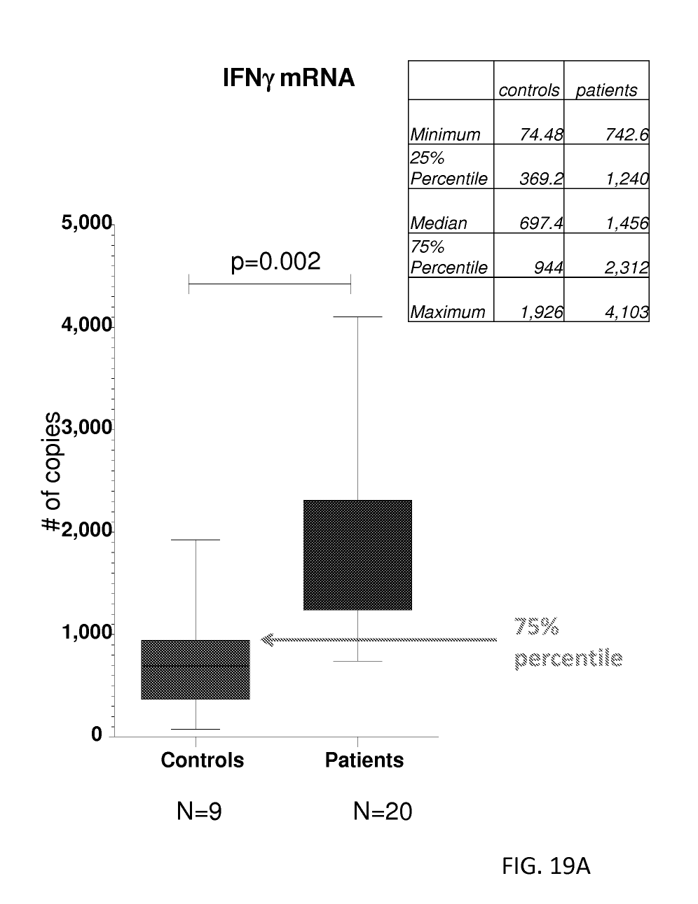

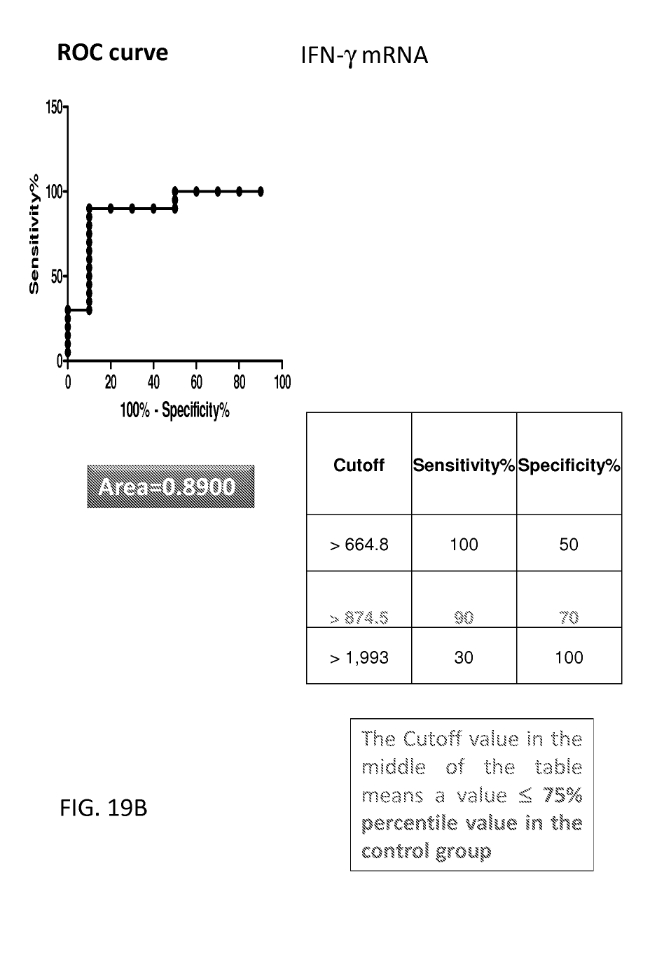

FIG. 19A shows diagnostic results for IFN-γ mRNA. FIG. 19B shows an exemplary ROC curve for IFN-γ mRNA.

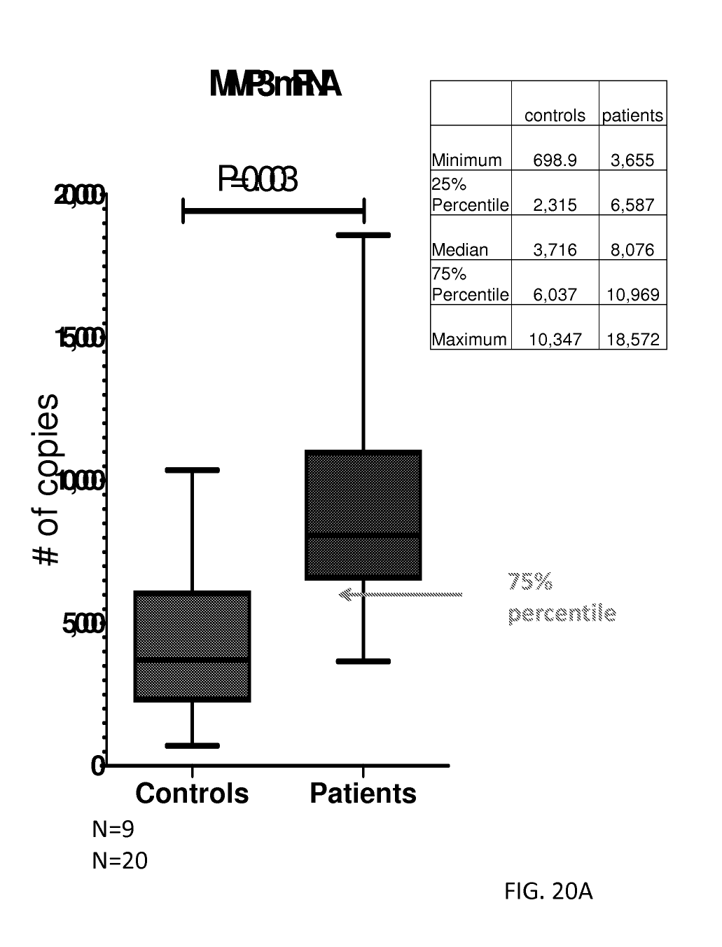

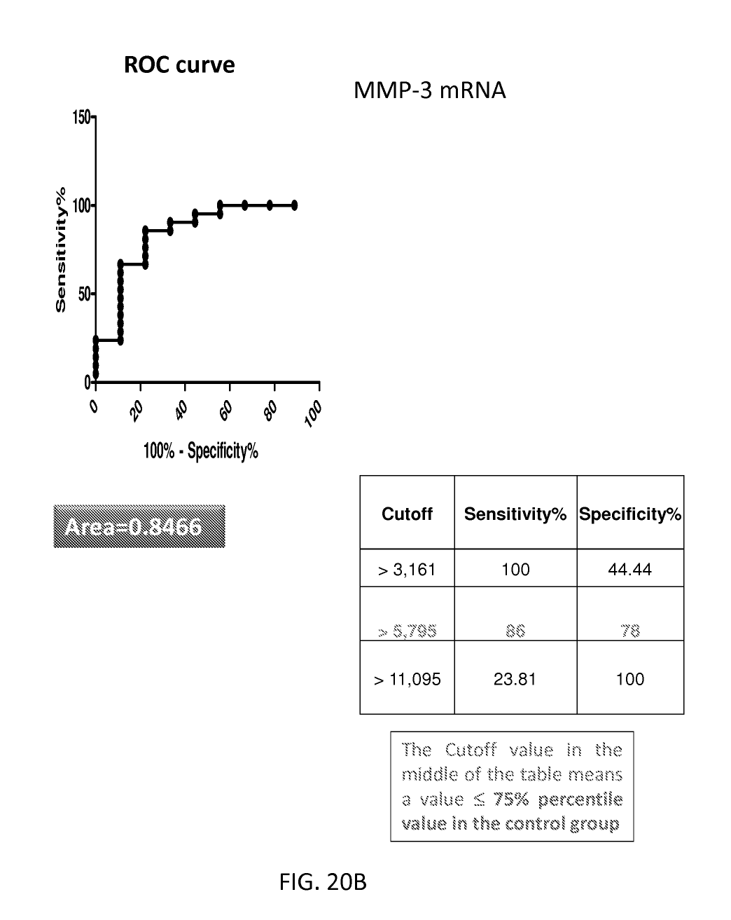

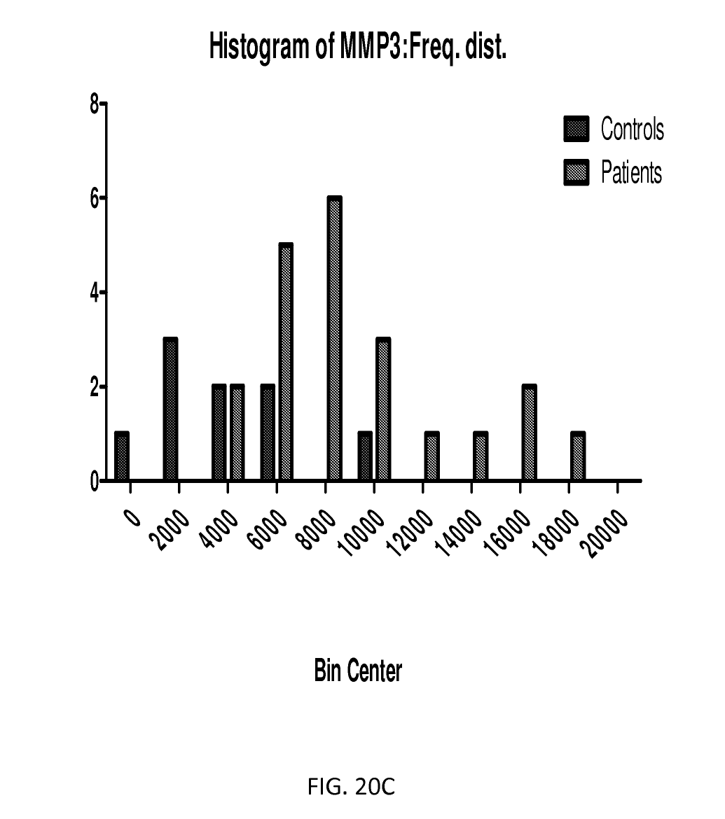

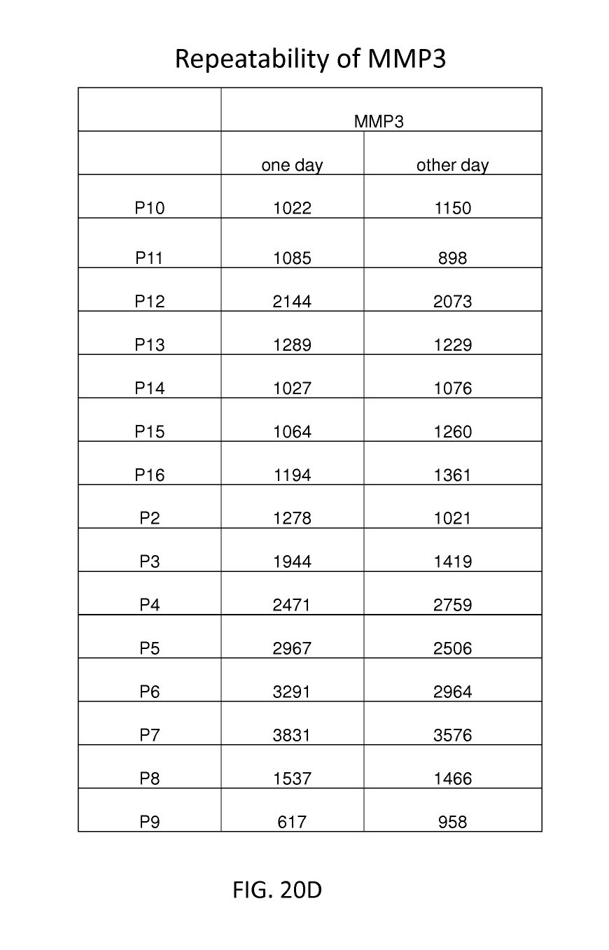

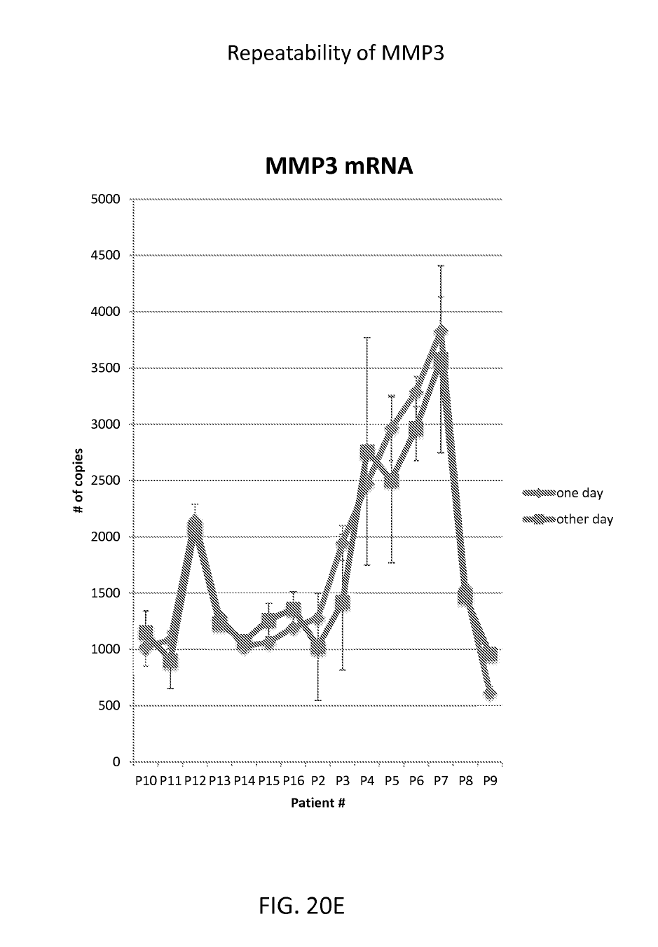

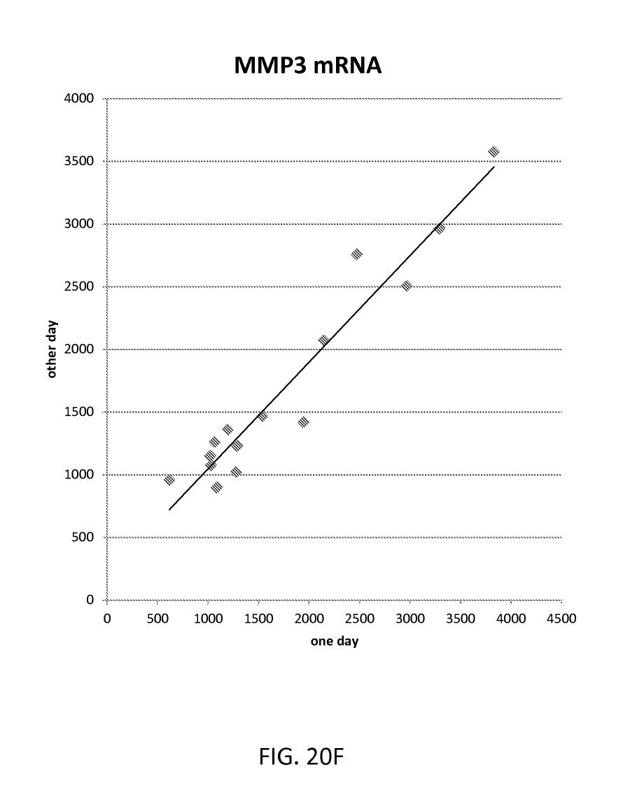

FIG. 20A shows diagnostic results for MMP-3 mRNA. FIG. 20B shows an exemplary ROC curve for MMP-3 mRNA. FIG. 20C shows exemplary diagnostic results as a histogram of the frequency distribution of MMP-3 mRNA. FIG. 20D demonstrates the repeatability for testing MMP-3 levels in particular patients. FIG. 20E illustrates the repeatability for testing MMP-3 levels in particular patients as demonstrated by a line graph. FIG. 20F shows a scattergraph demonstrating the repeatability of utilizing MMP-3 levels in certain patients.

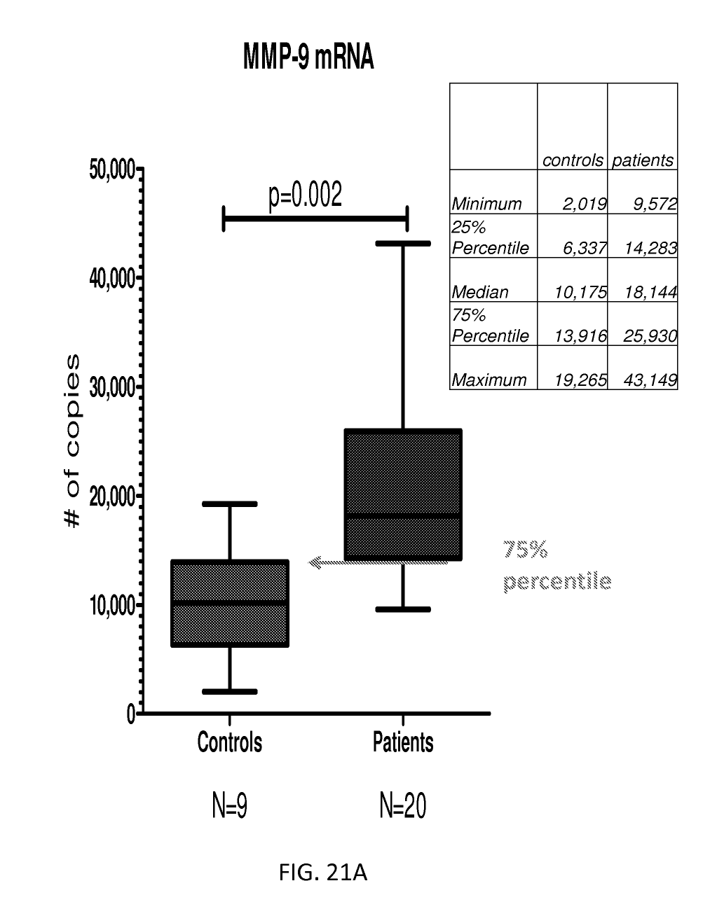

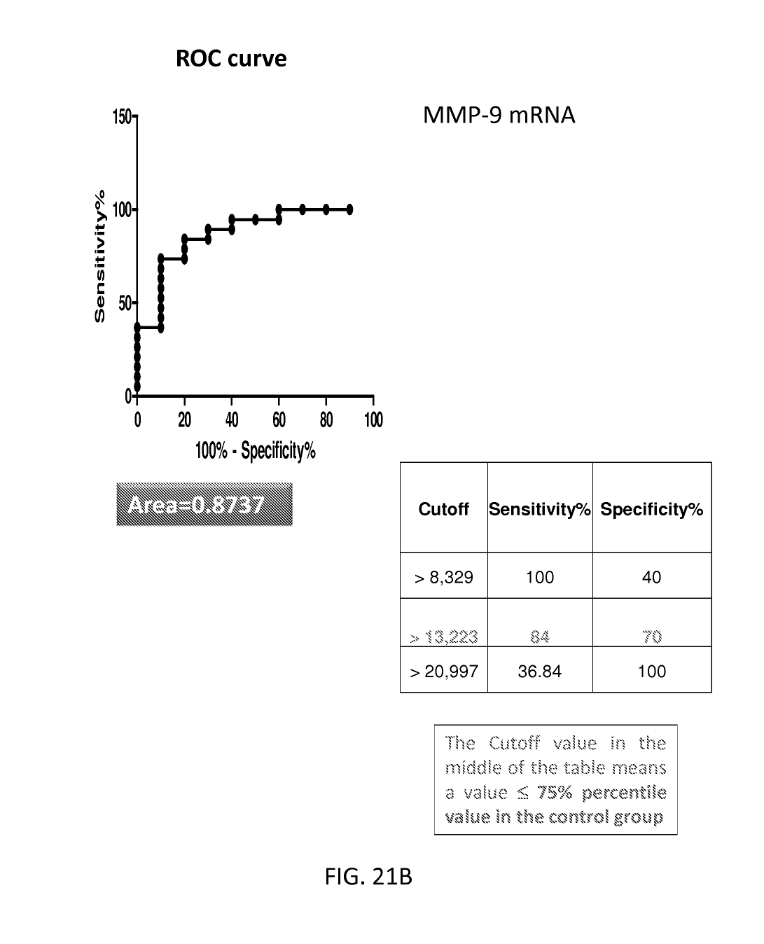

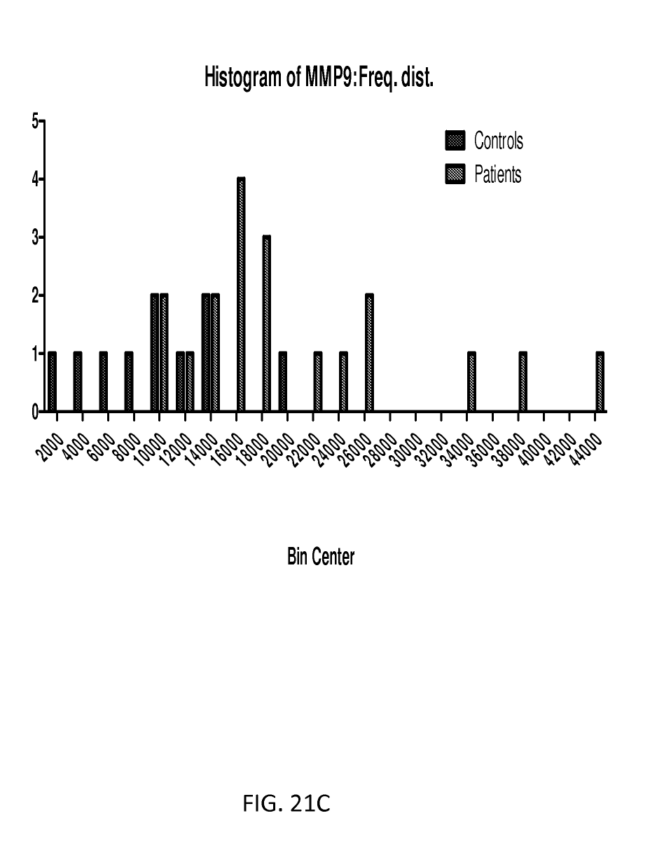

FIG. 21A shows diagnostic results for MMP-9 mRNA. FIG. 21B shows an exemplary ROC curve for MMP-9 mRNA. FIG. 21C shows exemplary diagnostic results as a histogram of the frequency distribution of MMP-9 mRNA.

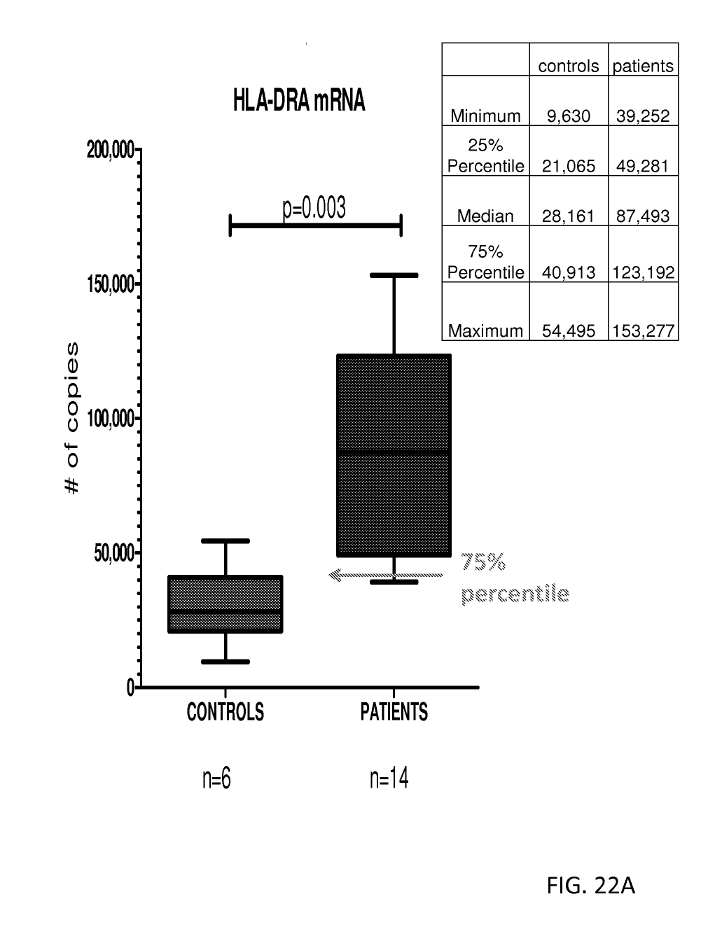

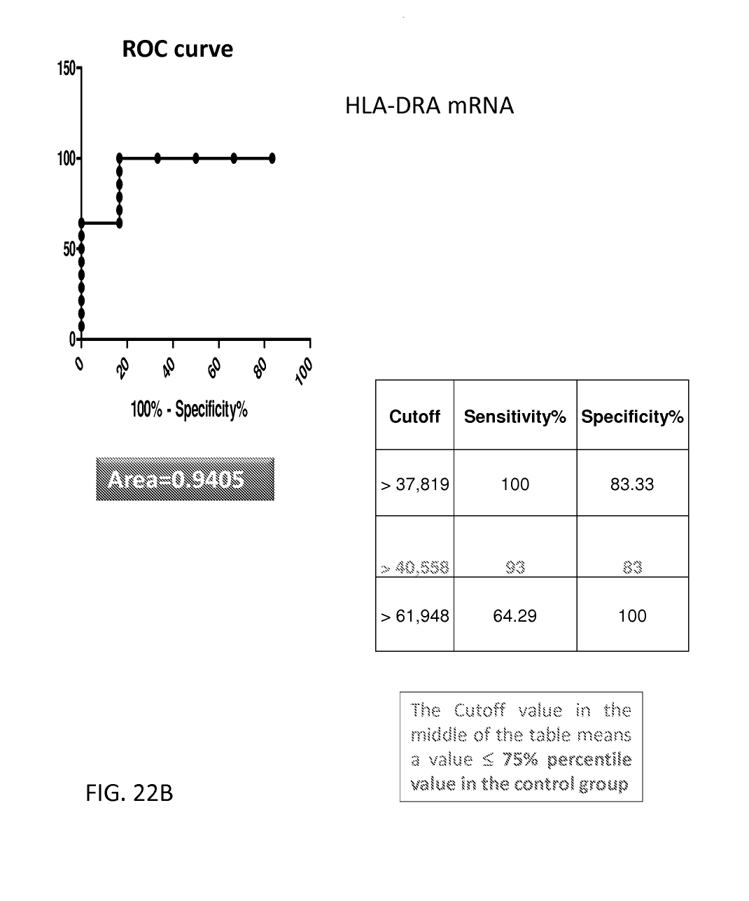

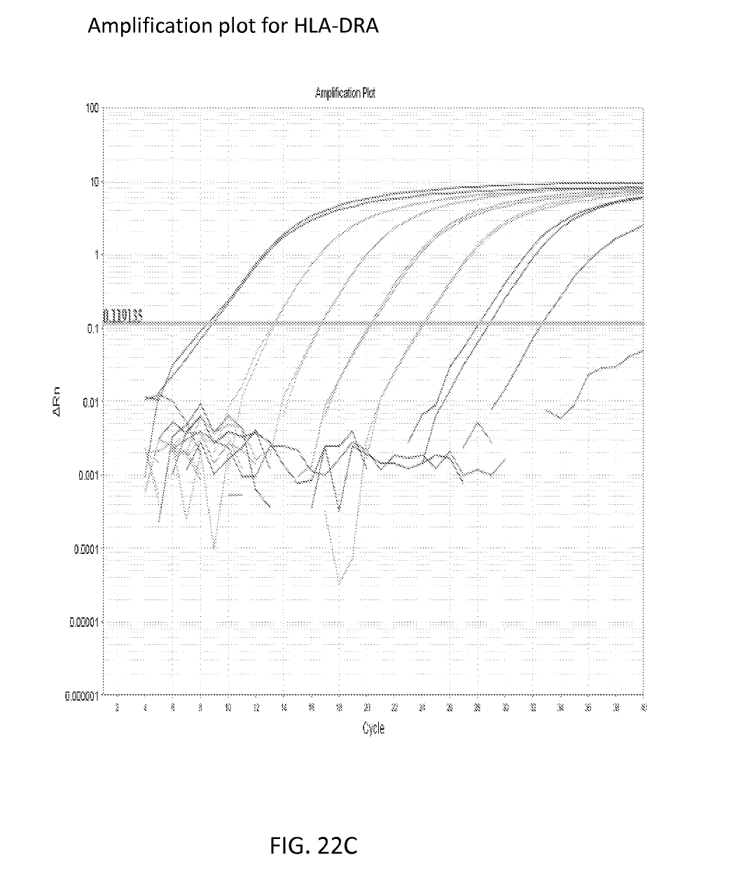

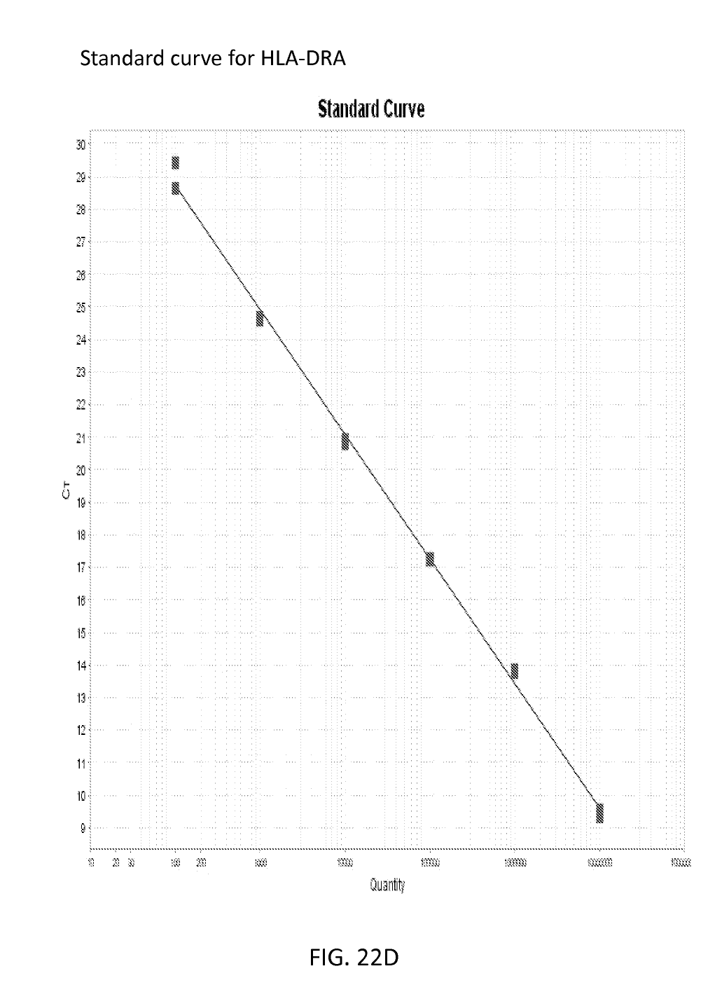

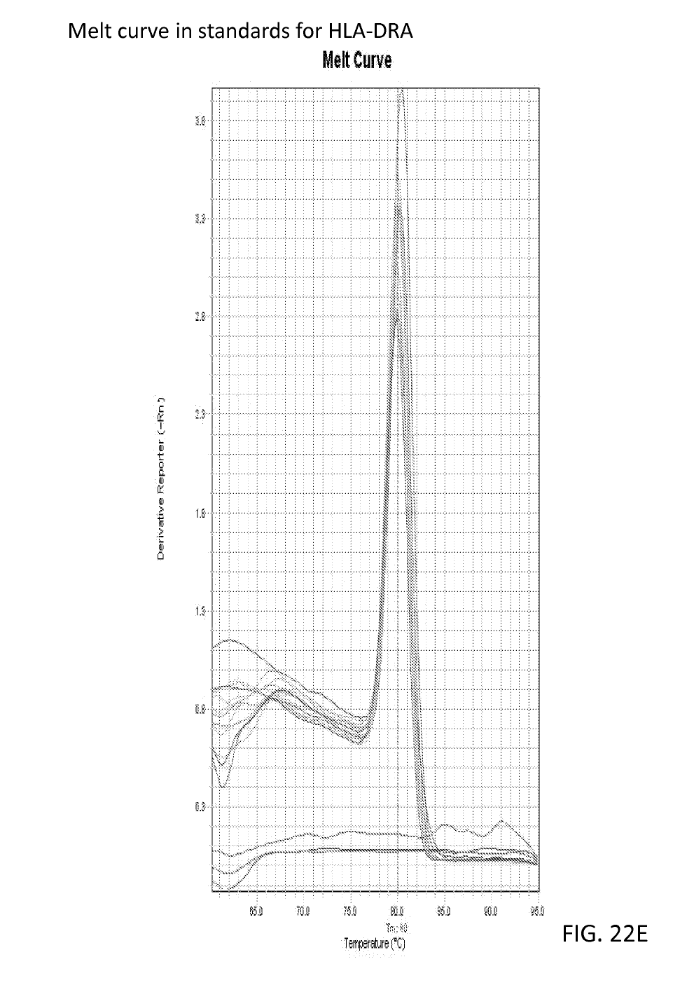

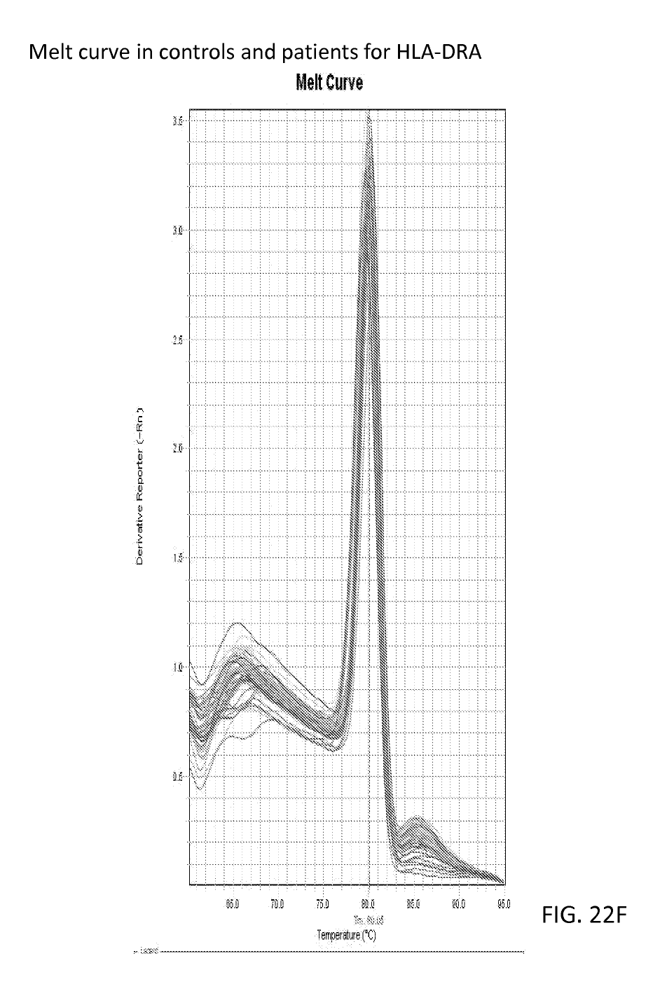

FIG. 22A shows diagnostic results for HLA-DRA mRNA. FIG. 22B shows an exemplary ROC curve for HLA-DRA mRNA. FIG. 22C shows an amplification plot for HLA-DRA. FIG. 22D provides an exemplary standard curve for HLA-DRA. FIG. 22E shows an exemplary melt curve in standards for HLA-DRA. FIG. 22F provides an exemplary melt curve in controls and patients for HLA-DRA.

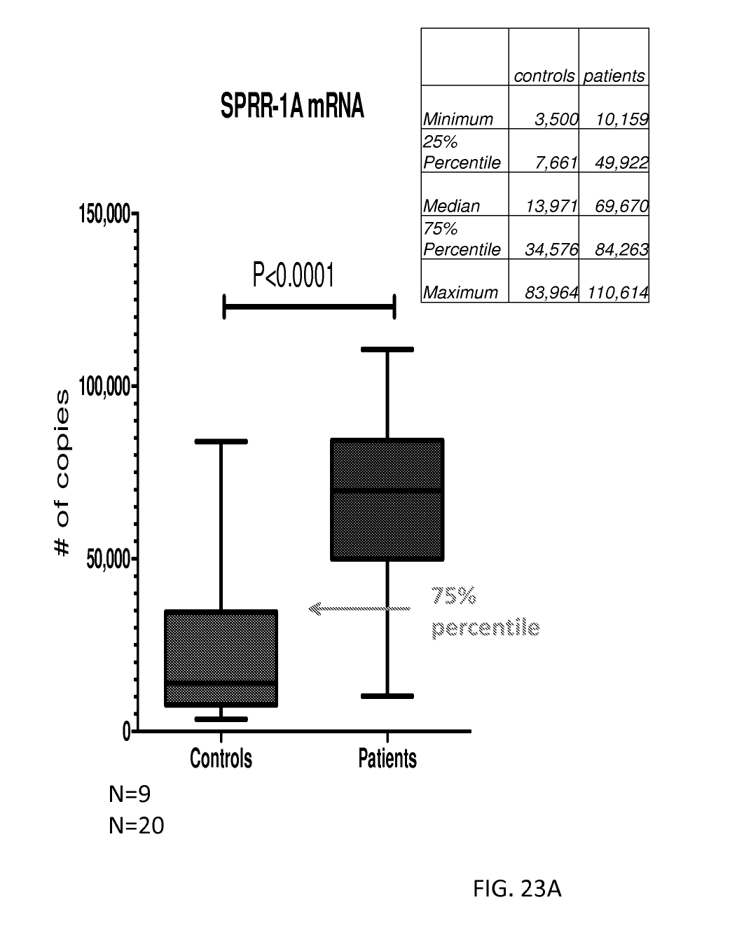

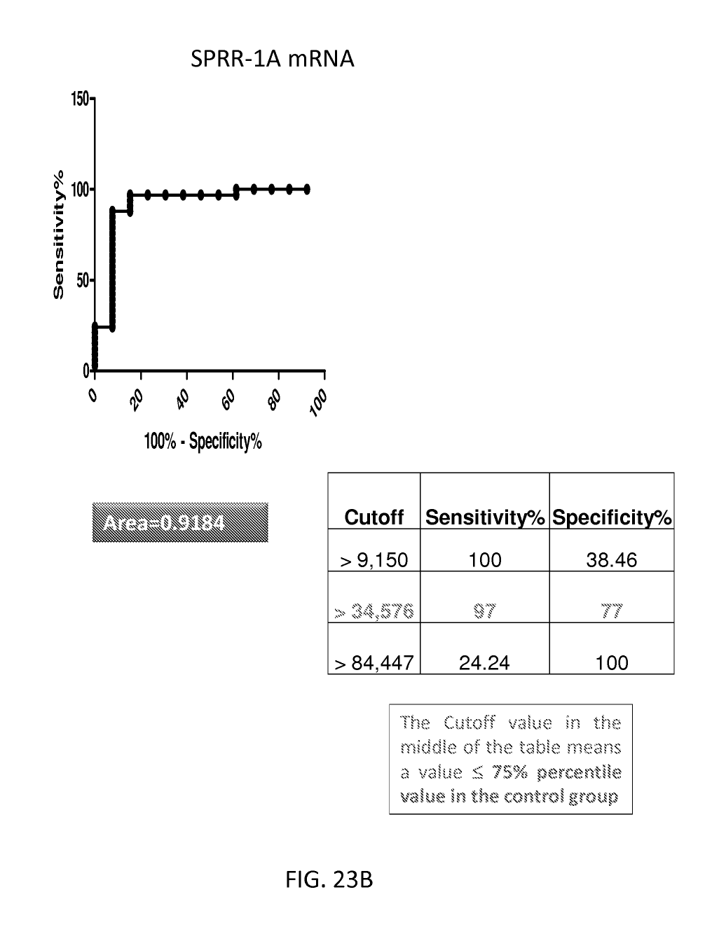

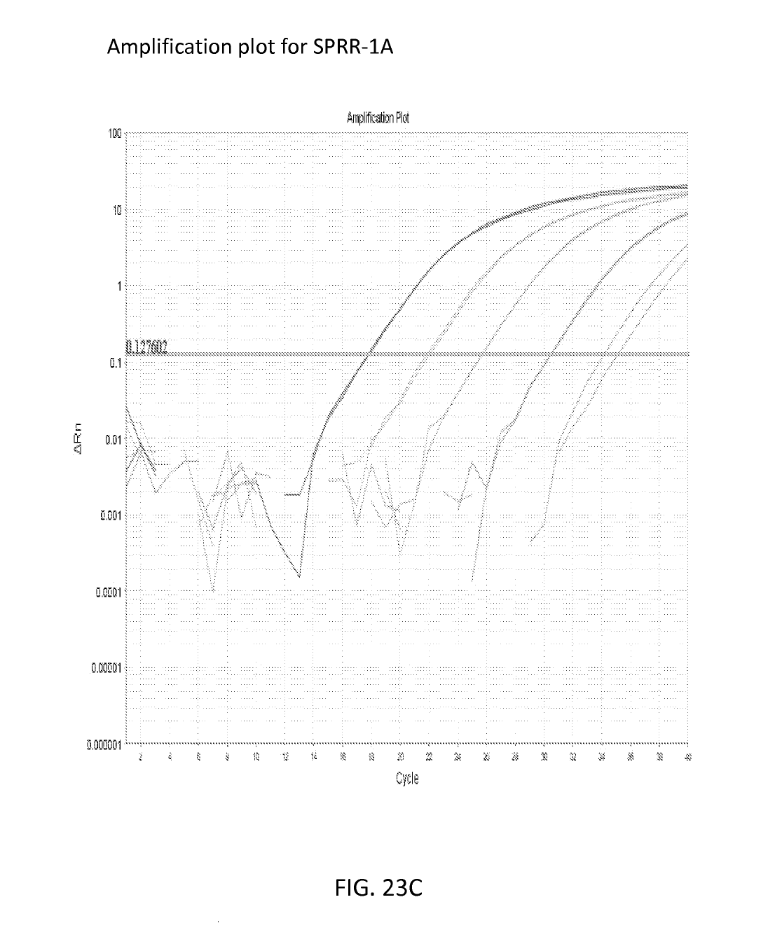

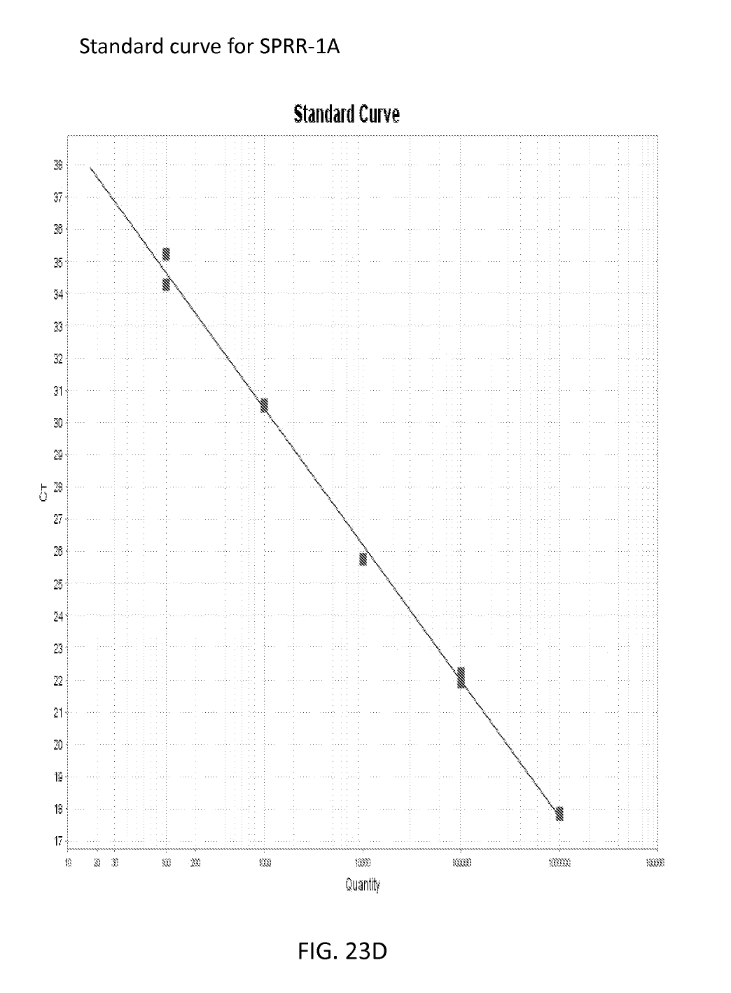

FIG. 23A shows diagnostic results for SPRR-1A mRNA. FIG. 23B shows an exemplary ROC curve for SPRR-1A mRNA. FIG. 23C shows an exemplary amplification plot for SPRR-1A. FIG. 23D shows an exemplary standard curve for SPRR-1A.

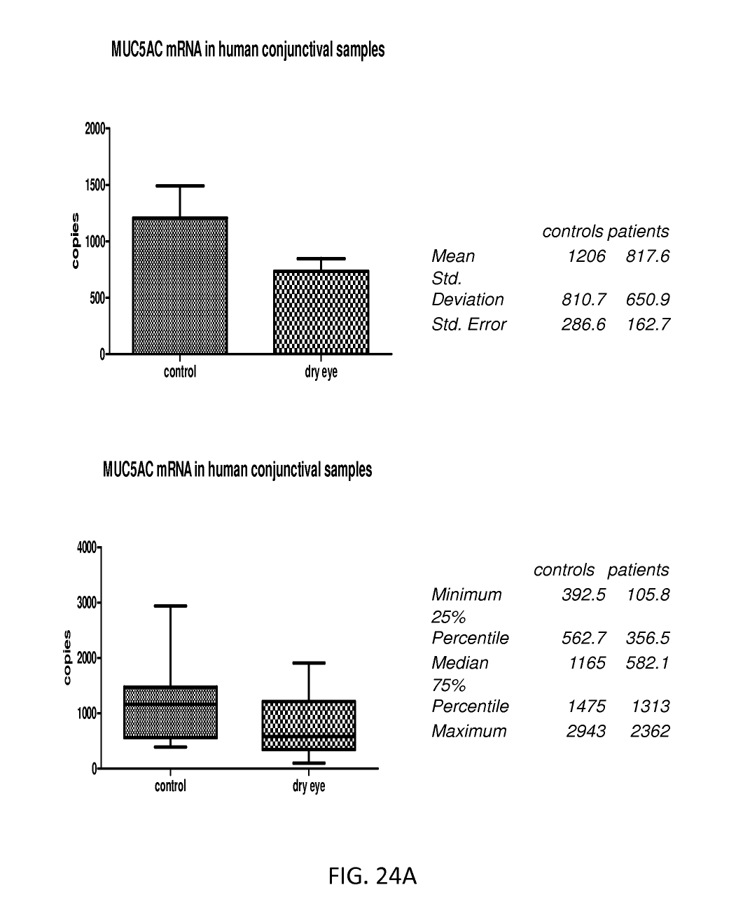

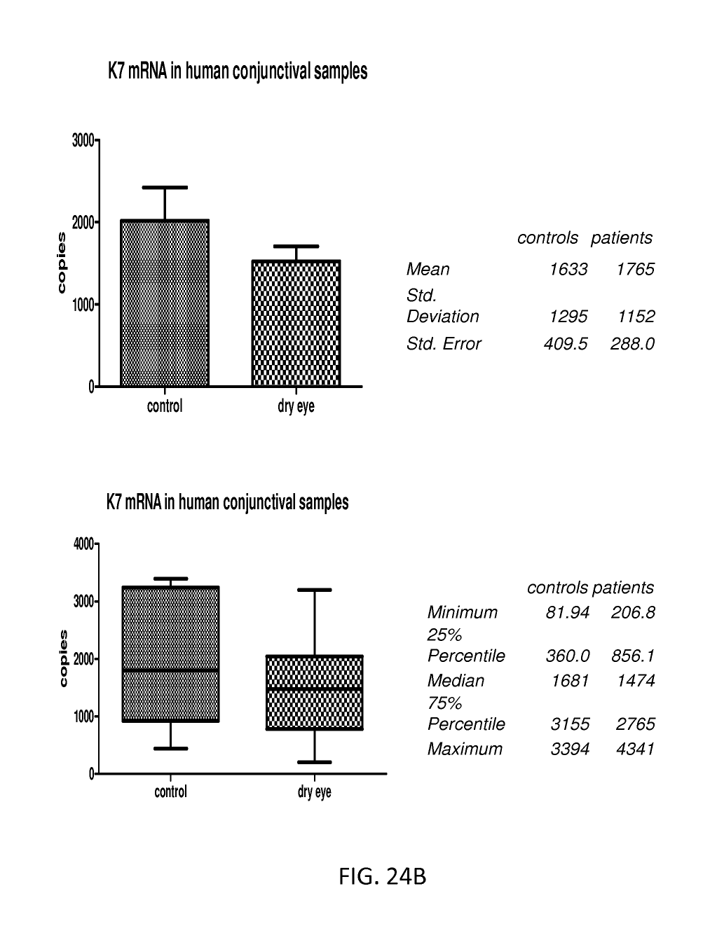

FIGS. 24A and 24B demonstrate exemplary copy numbers of MUC5AC mRNA (FIG. 24A) in human conjunctival samples and (cytokeratin 7) K7 mRNA in human conjunctival samples (FIG. 24B).

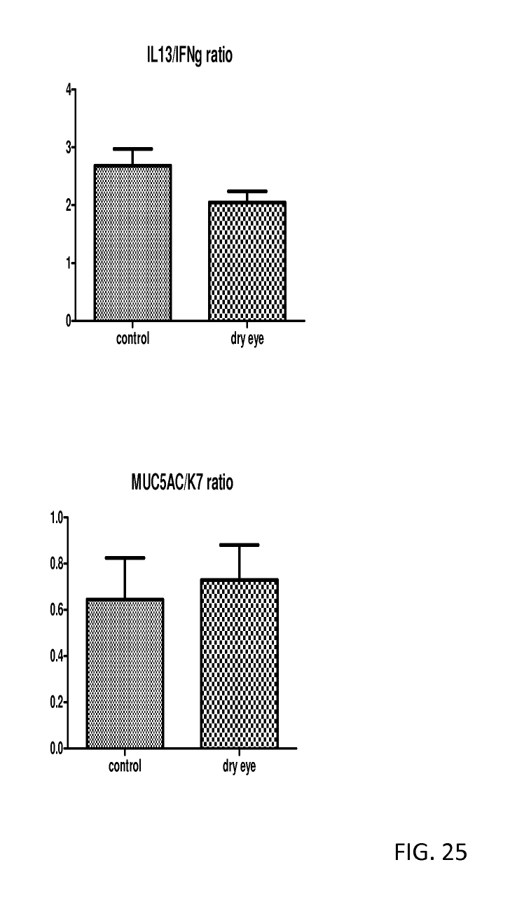

FIG. 25 demonstrates the ratio of expression of IL-13/IFN-γ and the ratio of MUC5AC/K7.

Exemplary quality control measures are provided in FIGS. 26 and 27. FIG. 26 illustrates the exemplary control HPRT-1 mRNA between controls and patients. FIG. 27 illustrates the exemplary control 18S mRNA between controls and patients.

Table 3 summarizes the exemplary diagnostic genes by area under their respective ROC curves.

TABLE 3
Diagnostic Genes sorted by area under ROC curve

| | p value | Area | | | |
|---|---|---|---|---|---|
| | Controls vs. | under | Cutoff | Sensi- | Speci- |
| GENE | Patients | ROC curve | value | tivity % | ficity % |
| IL-17A | 0.0004 | 0.9735 | >351.2 | 100 | 67 |
| | | | >437.3 | 95 | 78 |
| | | | >564.6 | 90 | 100 |
| HLRA- | 0.0030 | 0.9450 | >37,819 | 100 | 83 |
| DRA | | | | | |
| | | | >40,558 | 93 | 83 |
| | | | >61,948 | 64 | 100 |
| IL-6 | 0.0020 | 0.9350 | >674.6 | 100 | 80 |
| | | | >632.6 | 100 | 70 |
| | | | >1,152 | 45 | 100 |
| SPRR-1A | <0.0001 | 0.9184 | >9,150 | 100 | 38 |
| | | | >34,576 | 97 | 77 |
| | | | >84,447 | 24 | 100 |
| IFN-g | 0.0020 | 0.8900 | >664.8 | 100 | 50 |
| | | | >874.5 | 90 | 70 |
| | | | >1,993 | 30 | 100 |
| MMP-9 | 0.0020 | 0.8737 | >8,329 | 100 | 40 |
| | | | >13,223 | 84 | 70 |
| | | | >20,997 | 37 | 100 |
| MMP-3 | 0.0032 | 0.8466 | >3,161 | 100 | 44 |
| | | | >5,795 | 86 | 78 |
| | | | >11,095 | 24 | 100 |

In embodiments of the invention, the methods are therapeutic in that the results of the gene expression analysis are employed in the decision for appropriate treatment. For example, as shown in Table 4, when IL-17A is elevated, one can, in some embodiments, provide the individual with a T cell immunosuppressant, such as Cyclosporin.

TABLE 4
Value of Embodiments in Therapeutic Decision Making

| Elevated | |
|---|---|
| Biomarker | Indicated Therapy |
| IL-17A | T cell immunosuppressant such as Cyclosporin (Restasis ®) |
| IFN-gamma | T cell immunosuppressant such as Cyclosporin (Restasis ®) |
| MMP-3 | Antiprotease (corticosteroid or tetracycline) |
| MMP-9 | Antiprotease (corticosteroid or tetracycline) |
| IL-6 | Anti-inflammatory (cyclosporin, corticosteroid, omega-3,6 |
| | supplement) |
| HLA-DR | Anti-inflammatory (cyclosporin, corticosteroid, omega-3,6 |
| | supplement) |

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### PUBLICATIONS

Akpek E K, Merchant A, Pinar V, Foster C S. Ocular rosacea: patient characteristics and follow-up. Ophthalmology 1997; 104(11):1863-1867.
Altinors D D, Akça S, Akova Y A, et al. Smoking associated with damage to the lipid layer of the ocular surface. Am J Ophthalmol 2006; 141(6):1016-1021.
Behrens A, Doyle J J, Stern L, et al; Dysfunctional Tear Syndrome Study Group. Dysfunctional tear syndrome: a Delphi approach to treatment recommendations. Come 2006; 25(8):900-907.
Chun Y S, Kim J C. Treatment of superior limbic keratoconjunctivitis with a large-diameter contact lens and Botulium Toxin A. Cornea 2009; 28(7):752-758.
De Paiva C S, Corrales R M, Villarreal A L, et al. Apical corneal barrier disruption in experimental murine dry eye is abrogated by methylprednisolone and doxycycline. Invest Ophthalmol Vis Sci 2006; 47(7):2847-2856.
Ecoiffier T, El Annan J, Rashid S, Schaumberg D, Dana R. Modulation of integrin alpha4beta1 (VLA-4) in dry eye disease. Arch Ophthalmol 2008; 126(12):1695-1699.
Goyal S, Chauhan S K, Zhang Q, Dana R. Amelioration of murine dry eye disease by topical antagonist to chemokine receptor 2. Arch Ophthalmol 2009; 127(7):882-887.
Kojima T, Ishida R, Dogru M, et al. The effect of autologous serum eyedrops in the treatment of severe dry eye disease: a prospective randomized case-control study. Am J Ophthalmol 2005; 139(2):242-246.
Li D Q, Chen Z, Song X J, Luo L, Pflugfelder S C. Stimulation of matrix metalloproteinases by hyperosmolarity via a JNK pathway in human corneal epithelial cells. Invest Ophthalmol Vis Sci 2004; 45(12):4302-4311.
Mackie I A. Successful management of three consecutive cases of recurrent corneal erosion with botulinum toxin injections. Eye (Lond) 2004; 18(7):734-737.
Marsh P, Pflugfelder S C. Topical non-preserved methylprednisolone therapy of keratoconjunctivitis sicca. Ophthalmology 1999; 106(4):811-816.
Miljanovic' B, Trivedi K A, Dana M R, Gilbard J P, Buring J E, Schaumberg D A. Relation between dietary n-3 and n-6 fatty acids and clinically diagnosed dry eye syndrome in women. Am J Clin Nutr 2005; 82(4):887-893.
Moss S E, Klein R, Klein B E. Prevalence of and risk factors for dry eye syndrome. Arch Ophthalmol 2000; 118(9): 1264-1268.
Moss S E, Klein R, Klein B E. Incidence of dry eye in an older population. Arch Ophthalmol 2004; 122(3):369-373.
Pflüger O K. Zur Ernährung der cornea. Klin Monatsbl Augenheilkd 1882; 20:69-81. De Paiva C S, Raince J K, McClellan A J, et al. Homeostatic control of conjunctival mucosal goblet cells by NKTderived IL-13. Mucosal Immunol 2011; 4(4):397-408.
Pflugfelder S C, Maskin S L, Anderson B, et al. A randomized, double-masked, placebo-controlled, multicenter comparison of loteprednol etabonate ophthalmic suspension, 0.5%, and placebo for treatment of keratoconjunctivitis sicca in patients with delayed tear clearance. Am J Ophthalmol 2004; 138(3):444-457.
Rashid S, Jin Y, Ecoiffier T, Barabino S, Schaumberg D A, Dana M R. Topical omega-3 and omega-6 fatty acids for treatment of dry eye. Arch Ophthalmol 2008; 126(2):219-225.
Romero-Rangel T, Stavrou P, Cotter J, Rosenthal P, Baltatzis S, Foster CS. Gas-permeable scleral contact lens therapy in ocular surface disease. Am J Ophthalmol 2000; 130(1):25-32.
Rosenthal P, Croteau A. Fluid-ventilated, gas-permeable scleral contact lens is an effective option for managing severe ocular surface disease and many corneal disorders that would otherwise require penetrating keratoplasty. Eye Contact Lens 2005; 31(3):130-134.
Sall K, Stevenson O D, Mundorf T K, Reis B L; CSA Phase 3 Study Group. Two, multicenter, randomized studies of the efficacy and safety of cyclosporin ophthalmic emulsion in moderate to severe dry eye. Ophthalmology 2000; 107(4): 631-639.
Schaumberg D A, Sullivan D A, Buring J E, Dana M R. Prevalence of dry eye syndrome among US women. Am J Ophthalmol 2003; 136(2):318-326.
Schaumberg D A, Dana R, Buring J E, Sullivan D A. Prevalence of dry eye disease among US men: estimates from the Physicians' Health Studies. Arch Ophthalmol 2009(6);127:763.
Shimmura S, Shimazaki J, Tsubota K. Results of a population-based questionnaire on the symptoms and lifestyles associated with dry eye. Cornea 1999; 18(4):408-411.
Smith V A, Khan-Lim D, Anderson L, Cook S D, Dick A D. Does orally administered doxycycline reach the tear film? Br J Ophthalmol 2008; 92(6):856-859.
Uchino M, Schaumberg D A, Dogru M, et al. Prevalence of dry eye disease among Japanese visual display terminal users. Ophthalmology 2008; 115(11):1982-1988.
Uchiyama E, Aronowicz J D, Butovich I A, McCulley J P. Increased evaporative rates in laboratory testing conditions simulating airplane cabin relative humidity: an important factor for dry eye syndrome. Eye Contact Lens 2007; 33(4): 174-176.
Yoon, K.-C., Im, S.-K., Kim, G.-G., You, I.-C., Usefulness of double vital staining with 1% fluorescein and 1% lissamine green in patients with dry eye syndrome. Cornea 2011; 30:972-976
Zhang C, Zhang J, Yang B, Wu C. Cyclosporin A inhibits the production of IL-17 by memory Th17 cells from healthy individuals and patients with rheumatoid arthritis. Cytokine 2008; 42(3):345-352.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of identifying an individual that has, or is at risk or having, ocular surface inflammation, comprising the step of obtaining conjunctival cells in vivo from the individual and assaying expression level of one or more genes from the cells.

2. The method of claim 1, wherein the expression level is further defined as the copy number of the one or more expressed genes.

3. The method of claim 1, wherein the obtaining step is further defined as contacting the surface of the eye with a substrate.

4. The method of claim 3, wherein the substrate is a membrane.

5. The method of claim 1, wherein the one or more genes are selected from the group consisting of matrix metalloproteinase (MMP) 3, MMP9, interferon (IFN)γ, interleukin (IL)17A, IL-6, HLA class II histocompatibility antigen, DR alpha chain precursor (HLD-DRA), mucin (MUC5AC), keratin (K)7, IL-13, small proline rich (SPRR)-1A, and a combination thereof.

6. The method of claim 1, wherein the ocular surface inflammation is tear dysfunction, Sjögren's syndrome aqueous tear deficiency, non-Sjögren's syndrome tear dysfunction, meibomian gland disease/dysfunction, posterior blepharitis, delayed tear clearance, or medication toxicity.

7. The method of claim 1, further defined as assaying the expression level of two or more, three or more, four or more, five or more, six or more, seven or more, eight or more, or all of the genes selected from the group consisting of MMP3, MMP9, IFNγ, IL17A, IL-6, HLD-DRA, MUC5AC, K7, IL-13, and a combination thereof.

8. A method of identifying an individual that has, or is at risk of having, ocular surface inflammation, comprising the step of assaying the expression level of one or more genes selected from the group consisting of MMP3, MMP9, IFNγ, IL17A, IL-6, HLD-DRA, MUC5AC, K7, IL-13, and a combination thereof from a sample from the individual.

9. The method of claim 8, wherein the expression level is further defined as the copy number of the one or more expressed genes.

10. The method of claim 8, wherein the expression level is determined by mRNA level, protein level, or both.

11. The method of claim 8, further defined as assaying the expression level of two or more, three or more, four or more, five or more, six or more, seven or more, eight or more, or all of the genes selected from the group consisting of MMP3, MMP9, IFNγ, IL17A, IL-6, HLD-DRA, MUC5AC, K7, IL-13, and a combination thereof.

12. The method of claim 8, wherein when the expression level of the one or more genes selected from the group consisting of MMP3, MMP9, IFNγ, IL17A, IL-6, HLD-DRA, MUC5AC, K7, IL-13, and a combination thereof is elevated compared to a reference, the individual has, or is at risk for having, ocular surface inflammation.

13. The method of claim 1 or 8, wherein the individual is subjected to another method of identifying if the individual has or is at risk for having ocular surface inflammation.

14. The method of claim 1 or 8, further comprising the step of obtaining a sample from the individual.

15. The method of claim 1 or 8, wherein when the individual is identified as having ocular surface inflammation, the individual is subjected to one or more treatments for ocular surface inflammation.

16. A kit for assaying an individual for the presence or risk for ocular surface inflammation, comprising one or more reagents suitable for determining gene expression level of one or more genes selected from the group consisting of MMP3, MMP9, IFNγ, IL17A, IL-6, HLD-DRA, MUC5AC, K7, IL-13, and a combination thereof, wherein said reagents are housed in a suitable container.

17. A method of treating an individual for ocular surface inflammation, comprising the steps of providing a therapeutically effective amount of a suitable treatment for the ocular surface inflammation to the individual, said treatment resultant upon determination of expression level of one or more genes selected from the group consisting of MMP3, MMP9, IFNγ, IL17A, IL-6, HLD-DRA, MUC5AC, K7, IL-13, and a combination thereof.

18. The method of claim 17, wherein when the expression level of IL-17A is elevated compared to a reference, the individual is treated with a T cell immunosuppressant.

19. The method of claim 17, wherein when the expression level of IFN-γ is elevated compared to a reference, the individual is treated with a T cell immunosuppressant.

20. The method of claim 17, wherein when the expression level of MMP3 is elevated compared to a reference, the individual is treated with an antiprotease.

21. The method of claim 17, wherein when the expression level of MMP9 is elevated compared to a reference, the individual is treated with an antiprotease.

22. The method of claim 17, wherein when the expression level of IL-6 is elevated compared to a reference, the individual is treated with an anti-inflammatory.

23. The method of claim 17, wherein when the expression level of HLA-DRA is elevated compared to a reference, the individual is treated with an anti-inflammatory.

24. The method of claim 17, wherein the individual has one or more symptoms selected from the group consisting of dry eye, red eye, improper tear composition, decreased aqueous tear production, excessive tear evaporation, abnormality in lipid components or mucin in the tear production, tear instability, and a combination thereof.
